SEQUENCE LISTING (13 sequences)

SEQ ID NO: 1 - RNA, 40 nt, Artificial Sequence
  Synthetic Construct
ugcccucaacgccucguuccucccguccgagagcggaccu

SEQ ID NO: 2 - DNA, 15 bp, Artificial Sequence
  Synthetic Construct
ggctagctacaacga

SEQ ID NO: 3 - DNA, 33 bp, Artificial Sequence
  Synthetic Construct
cgggaggaaggctagctacaacgagaggcgttg

SEQ ID NO: 4 - DNA, 19 bp, Artificial Sequence
  Synthetic Construct
cgggaggaacgaggcgttg

SEQ ID NO: 5 - RNA, 21 nt, Artificial Sequence
  Synthetic Construct
aagucaugaaccacguuaaca

SEQ ID NO: 6 - RNA, 21 nt, Artificial Sequence
  Synthetic Construct
aagaacugcauggaccuaaca

SEQ ID NO: 7 - RNA, 19 nt, Artificial Sequence
  Synthetic Construct
cagcuucaugccuuuguaa

SEQ ID NO: 8 - RNA, 3135 nt, Mus musculus
cugagugugcgagagacagccuggcaggagagcgcucaggcagacagacagacagacggacggacuuggccaacccggucggccgcggacuccggacuguucauccguuugucuucauuuucucaccaacugcuuggauccagcgcccgcggcuccugcaccgguauuuuggggagcauuuggagagucccuucucccgccuuccacggagaagaagcucacaaguccgggcgcugcugacagcaucgagagcggcucccgaccgcgcgaggaaauaggcgagcggcuaccggccagcaacuuuccugacccagaggaccgguaacaaguggccgggagcgaacuuuugcaaaucucuucugcgccuuaaggcugccaccgagacuguaaagaaaagggagaagaggaaccuauacucauaccaguucgcacaggcggcugaaguugggcgagcgcuagccgcggcugccuagcgucccccucccccucacagcggaggaggggacaguuguuggaggccgggcggcagagcccgaucgcgggcuuccaccgagaauuccgugacgacuggucagcaccgccggagagccgcuguugcugggacuggucugcgggcuccaaggaaccgcugcuccccgagagcgcuccgugagugaccgcgacuuuucaaagcucggcaucgcgcgggagccuaccaacgugagugcuagcggagucuuaacccugcgcucccuggagcgaacuggggaggagggcucagggggaagcacugccgucuggagcgcacgcuccuaaacaaacuuuguuacagaagcagggacgcgcggguauccccccgcuucccggcgcgcuguugcggccccgaaacuucugcgcacagcccaggcuaaccccgcgugaagugacggaccguucuaugacugcaaagauggaaacgaccuucuacgacgaugcccucaacgccucguuccuccaguccgagagcggugccuacggcuacaguaacccuaagauccuaaaacagagcaugaccuugaaccuggccgacccggugggcagucugaagccgcaccuccgcgccaagaacucggaccuucucacgucgcccgacgucgggcugcucaagcuggcgucgccggagcuggagcgccugaucauccaguccagcaaugggcacaucaccacuacaccgacccccacccaguucuugugccccaagaacgugaccgacgagcaggagggcuucgccgagggcuucgugcgcgcccuggcugaacugcauagccagaacacgcuucccagugucaccuccgcggcacagccggucagcggggcgggcaugguggcucccgcgguggccucaguagcaggcgcuggcggcggugguggcuacagcgccagccugcacagugagccuccggucuacgccaaccucagcaacuucaacgcgggugcgcugagcagcggcgguggggcgcccuccuauggcgcggccgggcuggccuuucccucgcagccgcagcagcagcagcagccgccucagccgccgcaccacuugccccaacagaucccggugcagcacccgcggcugcaagcccugaaggaagagccgcagaccgugccggagaugccgggagagacgccgccccuguccccuaucgacauggagucucaggagcggaucaaggcagagaggaagcgcaugaggaaccgcauugccgccuccaagugccggaaaaggaagcuggagcggaucgcucggcuagaggaaaaagugaaaaccuugaaagcgcaaaacuccgagcuggcauccacggccaacaugcucagggaacagguggcacagcuuaagcagaaagucaugaaccacguuaacagugggugccaacucaugcuaacgcagcaguugcaaacguuuugagaacagacugucagggcugaggggcaauggaagaaaaaaaauaacagagacaaacuugagaacuugacugguugcgacagagaaaaaaaaaguguccgaguacugaagccaaggguacacaagauggacuggguugcgaccugacggcgcccccagugugcuggagugggaaggacguggcgcgccuggcuuuggcguggagccagagagcagcggccuauuggccggcagacuuugcggacgggcugugcccgcgcgcgaccagaacgauggacuuuucguuaacauugaccaagaacugcauggaccuaacauucgaucucauucaguauuaaaggggggugggagggguuacaaacugcaauagagacuguagauugcuucuguagugcuccuuaacacaaagcagggagggcugggaagggggggaggcuuguaagugccaggcuagacugcagaugaacuccccuggccugccucucucaacuguguauguacauauauauuuuuuuuuaauuugaugaaagcugauuacugucaauaaacagcuuccugccuuuguaaguuauuccauguuuguuuguuuggguguccugcccaguguuuguaaauaagagauuugaagcauucugaguuuaccauuuguaauaaaguauauaauuuuuuuauguuuuguuucugaaaauuuccagaaaggauauuuaagaaaauacaauaaacuauugaaaaguagcccccaaccucuuugcugcauuauccauagauaaugauagcuagaugaagugacagcugagugcccccaauauacuagggugaaagcuguguccccugucugauuuguaggaauagauacccgcaugcuaucauuggcucauacucucucccccggcaacacacaaguccagacuguacaccagaagauggugugguguuucuuaaggcuggaagaagggcuguugcaaggggagagggucagcccgcuggaaagcagacacuuugguugaaagcuguaugaaguggcaugugcugugaucauuuauaaucauaggaaagauuuaguaauuagcuguugauucucaaagcagggacccauggaaguuuuuaacaaaaggugucuccuuccaacuuugaaucugacaacuccuagaaaaagaugaccuuugcuugugcauauuuauaauagcguucguuaucacaauuaaauguauucaaau

SEQ ID NO: 9 - RNA, 3338 nt, Homo sapiens
gacaucaugggcuauuuuuagggguugacugguagcagauaaguguugagcucgggcuggauaagggcucagaguugcacugaguguggcugaagcagcgaggcgggaguggaggugcgcggagucaggcagacagacagacacagccagccagccaggucggcaguauaguccgaacugcaaaucuuauuuucuuuucaccuucucucuaacugcccagagcuagcgccuguggcucccgggcugguguuucgggaguguccagagagccuggucuccagccgcccccgggaggagagcccugcugcccaggcgcuguugacagcggcggaaagcagcgguacccacgcgcccgccgggggaagucggcgagcggcugcagcagcaaagaacuuucccggcugggaggaccggagacaaguggcagagucccggagcgaacuuuugcaagccuuuccugcgucuuaggcuucuccacggcgguaaagaccagaaggcggcggagagccacgcaagagaagaaggacgugcgcucagcuucgcucgcaccgguuguugaacuugggcgagcgcgagccgcggcugccgggcgcccccucccccuagcagcggaggaggggacaagucgucggaguccgggcggccaagacccgccgccggccggccacugcaggguccgcacugauccgcuccgcggggagagccgcugcucugggaagugaguucgccugcggacuccgaggaaccgcugcgcccgaagagcgcucagugagugaccgcgacuuuucaaagccggguagcgcgcgcgagucgacaaguaagagugcgggaggcaucuuaauuaacccugcgcucccuggagcgagcuggugaggagggcgcagcggggacgacagccagcgggugcgugcgcucuuagagaaacuuucccugucaaaggcuccggggggcgcgggugucccccgcuugccagagcccuguugcggccccgaaacuugugcgcgcagcccaaacuaaccucacgugaagugacggacuguucuaugacugcaaagauggaaacgaccuucuaugacgaugcccucaacgccucguuccucccguccgagagcggaccuuauggcuacaguaaccccaagauccugaaacagagcaugacccugaaccuggccgacccaguggggagccugaagccgcaccuccgcgccaagaacucggaccuccucaccucgcccgacguggggcugcucaagcuggcgucgcccgagcuggagcgccugauaauccaguccagcaacgggcacaucaccaccacgccgacccccacccaguuccugugccccaagaacgugacagaugagcaggagggcuucgccgagggcuucgugcgcgcccuggccgaacugcacagccagaacacgcugcccagcgucacgucggcggcgcagccggucaacggggcaggcaugguggcucccgcgguagccucgguggcagggggcagcggcagcggcggcuucagcgccagccugcacagcgagccgccggucuacgcaaaccucagcaacuucaacccaggcgcgcugagcagcggcggcggggcgcccuccuacggcgcggccggccuggccuuucccgcgcaaccccagcagcagcagcagccgccgcaccaccugccccagcagaugcccgugcagcacccgcggcugcaggcccugaaggaggagccucagacagugcccgagaugcccggcgagacaccgccccugucccccaucgacauggagucccaggagcggaucaaggcggagaggaagcgcaugaggaaccgcaucgcugccuccaagugccgaaaaaggaagcuggagagaaucgcccggcuggaggaaaaagugaaaaccuugaaagcucagaacucggagcuggcguccacggccaacaugcucagggaacagguggcacagcuuaaacagaaagucaugaaccacguuaacagugggugccaacucaugcuaacgcagcaguugcaaacauuuugaagagagaccgucgggggcugaggggcaacgaagaaaaaaaauaacacagagagacagacuugagaacuugacaaguugcgacggagagaaaaaagaaguguccgagaacuaaagccaaggguauccaaguuggacuggguugcguccugacggcgcccccagugugcacgagugggaaggacuuggcgcgcccucccuuggcguggagccagggagcggccgccugcgggcugccccgcuuugcggacgggcuguccccgcgcgaacggaacguuggacuuuucguuaacauugaccaagaacugcauggaccuaacauucgaucucauucaguauuaaaggggggagggggaggggguuacaaacugcaauagagacuguagauugcuucuguaguacuccuuaagaacacaaagcggggggaggguuggggaggggcggcaggagggagguuugugagagcgaggcugagccuacagaugaacucuuucuggccugccuucguuaacuguguauguacauauauauauuuuuuaauuugaugaaagcugauuacugucaauaaacagcuucaugccuuuguaaguuauuucuuguuuguuuguuuggguauccugcccaguguuguuuguaaauaagagauuuggagcacucugaguuuaccauuuguaauaaaguauauaauuuuuuuauguuuuguuucugaaaauuccagaaaggauauuuaagaaaauacaauaaacuauuggaaaguacuccccuaaccucuuuucugcaucaucuguagauacuagcuaucuagguggaguugaaagaguuaagaaugucgauuaaaaucacucucagugcuucuuacuauuaagcaguaaaaacuguucucuauuagacuuuagaaauaaauguaccugauguaccugaugcuauggucagguuauacuccuccucccccagcuaucuauauggaauugcuuaccaaaggauagugcgauguuucaggaggcuggaggaagggggguugcaguggagagggacagcccacugagaagucaaacauuucaaaguuuggauuguaucaaguggcaugugcugugaccauuuauaauguuaguagaaauuuuacaauaggugcuuauucucaaagcaggaauugguggcagauuuuacaaaagauguauccuuccaauuuggaaucuucucuuugacaauuccuagauaaaaagauggccuuugcuuaugaauauuuauaacagcauucuugucacaauaaauguauucaaauaccaaaaaaaaaaaaaaaaa

SEQ ID NO: 10 - DNA, 34 bp, Artificial Sequence
  Synthetic Construct
cgggaggaaggctagctacaacgagaggcgttgn

SEQ ID NO: 11 - DNA, 34 bp, Artificial Sequence
  Synthetic Construct
gcgacgtgaggctagctacaacgagtggaggagn

SEQ ID NO: 12 - RNA, 19 nt, Artificial Sequence
  Synthetic Construct
gauuacuagccgucuuccu

SEQ ID NO: 13 - RNA, 19 nt, Artificial Sequence
  Synthetic Construct
cagcuuccugccuuuguaa

(19) United States
(12) Patent Application Publication
(10) Pub. No.: US 2011/0065772 A1
(43) Pub. Date: Mar. 17, 2011

(54) TREATMENT OF RHEUMATOID ARTHRITIS

(75) Inventor: Levon M. Khachigian, New South Wales (AU)

(73) Assignee: NEW SOUTH INNOVATIONS PTY LIMITED

(21) Appl. No.: 12/667,007

(22) PCT Filed: Jun. 29, 2007
(86) PCT No.: PCT/AU2007/000914

§ 371 Date: Nov. 22, 2010

(51) Int. Cl. A61K 31/711; A61K 31/713; A61K 31/7105; A61K 31/7088; A61P 29/00; A61P 19/02

(52) U.S. Cl. 514/44 A; 514/44 R

(57) ABSTRACT

The present invention provides a method for treating or inhibiting rheumatoid arthritis in a subject, the method comprising administering to the subject a therapeutically effective amount of a nucleic acid which decreases the level of c-Jun mRNA, c-Jun mRNA translation or nuclear accumulation or activity of c-Jun protein.

### FIELD OF THE INVENTION

The present invention relates to methods and compositions for treating or inhibiting rheumatoid arthritis by reducing c-Jun expression. In particular, the present invention relates to methods of treating or inhibiting rheumatoid arthritis by reducing c-Jun expression involving the use of nucleic acid agents such as DNAzymes, RNA interference (RNAi) including short-interfering RNAs (siRNA), antisense oligonucleotides and ribozymes.

### BACKGROUND OF THE INVENTION

Rheumatoid arthritis is a common and debilitating disease characterized by inflammation of the distal diarthroidial joints, that affects approximately 1% of the adult population worldwide. Inflammatory cell infiltration and synovial hyperplasia in these joints contribute to gradual degradation of cartilage and bone, resulting in loss of normal joint function.

Collagen antibody-induced arthritis (CAIA) is a simple mouse model of rheumatoid arthritis that can be used to address questions of pathogenic mechanisms and to screen candidate therapeutic agents. Arthritis is induced by the systemic administration of a cocktail of monoclonal antibodies that target various regions of collagen type II, which is one of the major constituents of articular cartilage matrix proteins, together with lipopolysaccharide (LPS). Administration of LPS after the antibody cocktail reduces the amount of monoclonal antibody required to induce arthritis (Terato et al. (1995) Autoimmunity 22, 137-147). The pathogenic features of the CAIA model have striking similarities with human rheumatoid arthritis, including synovitis with infiltration of polymorphonuclear and mononuclear cells, pannus formation, cartilage degradation and bone erosion (Staines & Wooley (1994) Br. J. Rheumatol. 33, 798-807; Holmdahl et al. (1989) APMIS 97, 575-584). CAIA is an extension of the classical collagen-induced arthritis (CIA) model, which has been used extensively in rats, mice and primates, and involves immunization with type II collagen in adjuvant (Williams et al. (2005) Int. J. Exp. Pathol. 86, 267-278). The commercial availability of a cocktail of four collagen antibodies provides a straightforward model that avoids the need for host generation of autoantibodies to type II collagen (epitopes F10, A2, D8 and D1). Three of the four monoclonal antibodies are directed to conserved auto-antigenic epitopes that are located within a 83-amino-acid fragment (LysC2, the smallest arthritogenic fragment of type II collagen, which corresponds to amino acids 291-374) of the CB11 region (the CNBR-digested fragment, corresponding to amino acids 124-403) of type II collagen. The fourth monoclonal antibody recognizes an epitope within the LysC1 region (amino acids 124-290) in CB11 (Terato et al. (1995) Autoimmunity 22, 137-147; Terato et al. (1992) J. Immunol. 148, 2103-2108). CAIA has also been induced with a cocktail of monoclonal antibodies that target other epitopes in collagen type II with strain-dependent disease penetrance, and increased susceptibility in males and with age (Nandakumar et al. (2003) Am. J. Pathol. 163, 1827-1837). Although it is known that collagen-II-specific monoclonal antibodies bind to normal joint cartilage surface (Holmdahl et al. (1991) Autoimmunity 10, 27-34; Mo et al. (1994) Scand. J. Immunol. 39, 122-130), the precise mechanisms that lead to inflammatory arthritis in CAIA are unclear. Recent studies have demonstrated the involvement of both innate and adaptive immunity in CAIA (Wang et al. (2006) J. Clin. Invest. 116, 414-421).

The monoclonal antibody-induced arthritis model offers several key advantages over conventional CIA. First, arthritis is induced within only a few days (Terato et al. (1995) Autoimmunity 22, 137-147; McCoy et al. (2002) J. Clin. Invest. 110, 651-658; Yumoto et al. (2002) Proc. Natl Acad. Sci. USA 99, 4556-4561) rather than the several weeks that are required to induce arthritis by immunization with type II collagen. Second, unlike the CIA model, which requires autoantibody generation, CAIA can be generated in a wider spectrum of mice, including gene-deficient mice, transgenic mice and strains that are resistant to classic CIA. Moreover, the CAIA model has a high uptake rate (Labasi et al. (2002) J. Immunol. 168, 6436-6445) and cohorts can be synchronized from the time of antibody injection.

The CAIA model has been used to shed key insights into the arthritogenic roles played by a number of factors, including α1- and α2-integrins, prostaglandin E2 receptors, osteopontin and matrix metalloproteinases. For example, de Fougerolles et al. (2000) J. Clin. Invest. 105, 721-729, delivered anti-σ1-integrin monoclonal antibodies or anti-α2-integrin monoclonal antibodies (250 μg) i.p. into Balb/c mice starting on day 0, with repeated administration every third day for the duration of the experiment. Both antibodies inhibited arthritis. Mice deficient in α1-integrin had a reduced arthritic score that was comparable to α1-integrin antibody-treated wild-type mice. Interestingly, neither injection of anti-collagen antibodies alone, nor injection of LPS alone, induced arthritis (de Fougerolles et al. (2000) J. Clin. Invest. 105, 721-729). McCoy et al. (2002) J. Clin. Invest. 110, 651-658 (2002) reduced both the severity and incidence of arthritis in EP4 receptor-deficient animals compared with wild-type animals. Similar observations were reported using this model by Labasi et al. in mice deficient in P2X7 receptor, a ligand-gated ion channel (Labasi et al. (2002) J. Immunol. 168, 6436-6445). Yumoto et al. showed that osteopontin deficiency decreased the extent of articular cartilage destruction, chondrocyte apoptosis and synovial angiogenesis (Yumoto et al. (2002) Proc. Natl. Acad. Sci. USA 99, 4556-4561). Using scanning electron microscopy, they demonstrated that the articular cartilage surface was smooth in saline-injected mice but was lost on the joint surface in wild-type mice with CAIA. Osteopontin-deficient mice on the collagen antibody/LPS regime had no morphologic evidence of erosion (Yumoto et al. (2002) Proc. Natl. Acad. Sci. USA 99, 4556-4561). Itoh et al., on the other hand, were surprised to find that MMP-2 knockout mice showed severe clinical and histologic arthritis compared with wild-type mice, whereas arthritis was reduced in MMP-9 knockouts (Itoh et al. (2002) J. Immunol. 169, 2643-2647). MMP-2/MMP-9 double-deficient mice showed no significant differences from wild-type mice (Itoh et al. (2002) J. Immunol. 169, 2643-2647). The ease, reproducibility and synchronicity of the CAIA model thus renders it an attractive system for increasing our understanding of the molecular and cellular events that underlie human rheumatoid arthritis, and provides a useful platform for the preclinical evaluation of anti-arthritic drugs and approaches.

c-Jun

Immediate-early genes, like the transcription factor c-Jun, control the expression of multiple regulatory genes and are, by definition, “master-regulators”. c-Jun is a member of the basic region-leucine zipper (bZIP) protein family that homodimerises and heterodimerises with other bZIP proteins to form the transcription factor, activating protein-1 (AP-1; Shaulian & Karin (2001) Oncogene 20: 2390-2400). c-Jun has been linked with cell proliferation, transformation, and apoptosis. For example, skin tumour promotion is blocked in mice expressing a dominant-negative transactivation mutant of c-Jun (Young et al. (1999). Proc. Natl. Acad. Sci. USA 96: 9827-9832). Microinjection of antibodies to c-Jun into Swiss 3T3 cells inhibits cell cycle progression (Kovary & Bravo (1991) Mol. Cell. Biol. 11: 4466-4472). Compared with primary fibroblasts cultured from wild-type littermates, primary fibroblasts cultured from live heterozygous and homozygous mutant c-Jun mouse embryos, which die in utero (Hilberg et al. (1993) Nature 365: 179-181; Johnson et al. (1993) Genes Dev. 7: 1309-1317), have greatly reduced growth rates in culture that cannot be overcome by the addition of mitogen (Johnson et al. (1993) Genes Dev. 7: 1309-1317). c-Jun has also been implicated in apoptosis. For example, c-Jun null mouse embryo fibroblasts are resistant to apoptosis induced by UVC radiation (Shaulian et al. (2000) Cell 103: 897-907). More recently, a direct link between c-Jun and the process of angiogenesis has been shown using a gene specific catalytic DNA (Zhang et al. (2004) Journal of National Cancer Institute 96: 683-96; Khachigian (2000) J. Clin. Invest. 106: 1189-1195).

Insights into the function of a given gene product in a complex biological process such as angiogenesis may be obtained using gene-targeting strategies that employ DNA enzymes (DNAzymes). DNAzymes are synthetic, all-DNA-based catalysts that can be engineered to bind their complementary sequences in their target messenger RNA (mRNA) through Watson-Crick base pairing and cleave the mRNA at predetermined phosphodiester linkages (Khachigian (2002) Curr. Opin. Mol. Therap. 4: 119-121). These catalysts have emerged as a potential new class of nucleic acid-based drugs because of their relative ease and low cost of synthesis and flexible rational design features. Gene-specificity of a DNAzyme for an mRNA is determined by the sequence of deoxyribonucleotides in the hybridising arms of the DNAzyme; the hybridising arms are generally seven or more nucleotides long (Schubert et al. (2003) Nucleic Acids Res 31: 5982-92). A “general purpose” DNAzyme comprising a 15-nucleotide cation-dependent catalytic domain (designated “10-23”) that cleaves the phosphodiester linkage between an unpaired purine and a paired pyrimidine in the target mRNA (Santoro & Joyce (1997) Proc. Natl. Acad. Sci. USA 94: 4262-4266) was developed using a systematic in vitro selection strategy. DNAzymes do not rely on RNase H for destruction of the mRNA; these agents are stable in serum (Dass et al. (2002) Antisense Nucleic Acid Drug Dev 12: 289-99; Santiago et al. (1999) Nature Med. 5: 1264-1269) and can be produced at relative low cost. DNAzyme stability can be further increased, without compromising catalytic efficiency, by incorporation of structural modifications (such as base inversions, methylene bridges, etc) into the molecule. DNAzymes targeting the immediate-early gene Egr-1 have been used to suppress numerous vascular pathologic settings, such as intimal thickening after carotid artery injury in rats (Lowe et al. (2002) Thromb. Haemost. 87: 134-140; Santiago et al. (1999) Nature Med. 5: 1264-1269), in-stent restenosis after stenting coronary arteries in pigs (Lowe et al. (2001) Circulation Research 89: 670-677), and more recently, tumour angiogenesis (Fahmy et al. (2003) Nature Med. 9: 1026-32).

The inventors have previously demonstrated the capacity of DNAzymes targeting the transcription factor c-Jun to inhibit proliferation of a variety of cell types, and also to promote disease progression in a wide spectrum of animal models, including arterial thickening following injury (Khachigian et al. (2002) J. Biol. Chem. 277, 22985-22991), angiogenesis (Zhang et al. (2004) J. Natl Cancer Instit. 96, 683-696) and tumor growth (Zhang et al. (2004) J. Natl Cancer Instit. 96, 683-696).

### SUMMARY OF THE INVENTION

The inventors have now shown that agents which target c-Jun also inhibit vascular leakiness, endothelial-monocytic-cell adhesion in vitro, leukocyte rolling, adhesion and extravasation in cytokine-challenged venules and lung inflammation after endotoxin exposure. Further the inventors have shown a therapeutic role for agents targeting c-Jun in an animal model of arthritis.

Accordingly, in a first aspect of the present invention there is provided a method of treating or inhibiting rheumatoid arthritis in a subject, the method comprising administering to the subject a therapeutically effective amount of a nucleic acid which decreases the level of c-Jun mRNA, c-Jun mRNA translation or nuclear accumulation or activity of c-Jun protein.

In a preferred embodiment of the present invention the nucleic acid is selected from the group consisting of a DNAzyme targeted against c-Jun, a c-Jun antisense oligonucleotide, a ribozyme targeted against c-Jun, and a ssDNA targeted against c-Jun dsDNA such that the ssDNA forms a triplex with the c-Jun dsDNA. In an alternative embodiment of the present invention the nucleic acid is dsRNA targeted against c-Jun mRNA, a nucleic acid molecule which results in production of dsRNA targeted against c-Jun mRNA or small interfering RNA molecules (siRNA) targeted against c-Jun mRNA.

In a second aspect of the present invention there is provided a pharmaceutical composition comprising a therapeutically effective amount of a nucleic acid which decreases the level of c-Jun mRNA, c-Jun mRNA translation or nuclear accumulation or activity of c-Jun protein, together with a pharmaceutically excipient, for treating or inhibiting arthritis.

### DESCRIPTION OF THE FIGURES

FIG. 1 shows Dz13 localizes to vascular endothelium and inhibits retinal neovascularization and vascular leakiness. (a) Dz13 inhibits retinal neovascularization in the retinopathy of prematurity model. Serial cross-sections of the eyes were stained with H&E and blood vessels in the retina were quantitated by light microscopy under 400× magnification and expressed as the mean±SEM. The figure shows localization of FITC-labeled DNAzyme or siRNA in retinal neovessels by fluorescence microscopy. (b) Dz13 inhibits vascular permeability induced by IgE-DNP in the passive cutaneous anaphylaxis model. The figure shows representative dye leakage and localization of FITC-labeled DNAzyme in blood vessels in ears by fluorescence microscopy (with corresponding H&E-stained sister section shown). (c) Dz13 inhibits VEGF₁₆₅-induced vascular permeability in the Miles assay. The figure shows time-dependent tissue accumulation of FITC-labeled DNAzyme and sister H&E-stained cross sections. (d) Intradermal injections of 100 μg Dz13 were performed in 6 w.o. female Balb/c nude mice. At 5 and 60 min, skin surrounding the injection site was resected, homogenized in 1.2 ml TRIzol and DNA extracted from skin tissue and column purified. The DNA was incubated with ³²P-5′-end labeled 40 nt RNA substrate (5′-UGC CCU CAA CGC CUC GUU CCU CCC GUC CGA GAG CGG ACC U-3; SEQ ID No:1) for 1 h at 37° C. Cleavage products were separated by electrophoresis on 12% PAGE denaturing gels and visualized by autoradiography. *denotes P<0.05 compared to control using Student's t-test or ANOVA.

FIG. 2 shows Dz13 inhibits cytokine-inducible monocytic cell-endothelial cell adhesion in vitro and inflammation in mesenteric microcirculation of rats. (a) HMEC-1 transfected with Dz13 or Dz13scr were incubated with IL-1beta prior to the addition of a suspension of THP-1 monocytic cells. Alternatively the THP-1 cells were transfected with DNAzyme. Fluorescence microscopy demonstrates that although THP-1 cells took up FITC-labeled DNAzyme, Dz13 failed to inhibit adhesion of the monocytic cells to endothelial cells. (b) HMEC-1 transfected with siRNA or siRNAscr were incubated with IL-1beta, then a suspension of THP-1 monocytic cells was added to each well. (c) Dz13 inhibits inflammation in the mesenteric venules of rats. Fluorescence microscopy on cross-sections of mesenteric venules demonstrates FITC-labeled DNAzyme uptake into the venular endothelium. *denotes P<0.05 compared to control using Student's t-test or ANOVA.

FIG. 3 shows Dz13 inhibits gene expression in mesenteric venular endothelium and microvascular endothelial cells. (a) Immunohistochemical analysis was performed for a variety of antigens in rat mesenteric venules (see Table 1 for blinded scoring data). Figure shows representative immunostaining for c-Jun and ICAM-1 (arrows) at 100× magnification. Hematoxylin counterstaining was omitted in the case of c-Jun to demonstrate predominant nuclear staining. (b) Western blot analysis of total extracts of microvascular endothelial cells exposed to 20 ng/ml IL-1beta for the times indicated using antibodies to c-Jun and ICAM-1 (left panel) and with extracts harvested 4 h after cytokine treatment with the antibodies indicated (right panel). Cells were transfected with 0.2 μM of Dz13 or Dz13scr. Coomassie blue gel indicates unbiased loading. (c) Scanning densitometric assessment of band intensity from Western blotting normalised to beta-Actin. (d) Dz13 inhibits neutrophil infiltration in lungs of LPS-challenged mice. Neutrophils in the bronchoalveolar fluid were resuspended in PBS and counted. The figure also shows representative H&E-stained cross-sections of paraffin-embedded lung in the 200 μg DNAzyme and control groups at 100× magnification. Fluorescence microscopy demonstrates FITC-DNAzyme localization in lung tissue. *denotes P<0.05 compared to control using Student's t-test or ANOVA.

FIG. 4 shows Dz13 inhibits joint thickness and synovial inflammatory cell infiltration in arthritic mice. (a) DNAzyme was administered intraarticularly into the hind paw joint of mice previously injected i.p. with a cocktail of 4 monoclonal antibodies to type II collagen and LPS. Hind paw thickness was determined using electronic Vernier callipers (panel above right). Quantitative assessment of area densities in the synovial lining of the tibiotarsal joint was performed under 200× magnification and a modified version of NIH Image software. Three random areas of synovial tissue on the medial aspect of the joint were assessed for each animal in a blinded fashion (panel below left). Semi-quantitative assessment bone erosion in the talus and distal tibia was made under 200× magnification and a modified tiered scoring criteria (panel below right). (b, c) Representative high power fields (400× and 600× magnification in b and c, respectively) showing proximal talus and distal tibia in control mice (No CAIA, for collagen antibody-induced arthritis) and the medial edge of tibia in the other groups in which collagen antibodies were administered. The talus (ta) and tibia (ti) in No CAIA mice has smooth epiphysis (ep) and cortical bone (cb) surfaces; the synovium (S) is also indicated. However, there is extensive erosion of bone on the surfaces of the distal tibia in the CAIA and CAIA+Dz13scr groups (arrows), but not in Dz13 animals. There are substantial differences in the inflammatory cell composition in synovial tissue between the treatment groups. Short and long arrows in (b) indicate modest and severe bone erosion, respectively. Fluorescence microscopy demonstrates FITC-DNAzyme localization within endothelium. Arrows in (c) indicate osteoclasts. The majority of cells in the Dz13 group are fibroblast-like synoviocytes (sy) and macrophages (ma) with limited number of neutrophils (ne) and osteoclasts (oc). On the contrary, a significant proportion of cells in the CAIA and CAIA+Dz13scr groups are neutrophils, with substantial infiltration by macrophages andosteoclasts but limited synoviocites. (d) Immunohistochemical analysis for c-Jun antigenicity in Dz13 treated joint (CAIA), lung sepsis and eye (ROP) models. St denotes stimulation (ie. hyperoxia/normoxia, collagen antibodies/LPS, or LPS). Arrows indicate c-Jun antigenicity. *denotes P<0.05 compared to control using Student's t-test or ANOVA.

FIG. 5 shows a sequence alignment between mouse and human c-Jun sequences. Sequence alignment between mouse and human c-Jun gene sequences. The figure includes a consensus sequence indicating the overall degree of homology.

### DETAILED DESCRIPTION OF THE INVENTION

The inventors have now shown that agents which target c-Jun also inhibit endothelial-monocytic-cell adhesion in vitro, leukocyte rolling, adhesion and extravasation in cytokine-challenged venules and lung inflammation after endotoxin exposure. Further the inventors have shown a positive role for agents targeting c-Jun in an animal model of arthritis. In particular, the inventors have demonstrated that a DNAzyme targeting c-Jun inhibited neutrophil accumulation in the synovium, inhibited neovascularization and joint thickening, blocked the accumulation of multi-nucleated osteoclast-like cells at the bone surface, and also reduced bone erosion.

Accordingly, in a first aspect of the present invention there is provided a method for treating or inhibiting rheumatoid arthritis in a subject, the method comprising administering to the subject a therapeutically effective amount of a nucleic acid which decreases the level of c-Jun mRNA, c-Jun mRNA translation or nuclear accumulation or activity of c-Jun protein.

In a preferred embodiment of the present invention the nucleic acid is selected from the group consisting of a DNAzyme targeted against c-Jun, a c-Jun antisense oligonucleotide, a ribozyme targeted against c-Jun, and a ssDNA targeted against c-Jun dsDNA such that the ssDNA forms a triplex with the c-Jun dsDNA. In an alternative embodiment of the present invention the nucleic acid is dsRNA targeted against c-Jun mRNA, a nucleic acid molecule which results in production of dsRNA targeted against c-Jun mRNA or small interfering RNA molecules targeted against c-Jun mRNA.

Although the subject may be animal or human, it is preferred that the subject is a human.

As will be recognised by those skilled in the relevant art there are a number of means by which the method of the present invention may be achieved.

In a preferred embodiment of the present invention the method is achieved by cleavage of c-Jun mRNA by a sequence specific DNAzyme. In a further preferred embodiment, the DNAzyme comprises:
(i) a catalytic domain which cleaves mRNA at a pruine:pyrimidine cleavage site;
(ii) a first binding domain contiguous with the 5′ end of the catalytic domain; and
(iii) a second binding domain contiguous with the 3′ end of the catalytic domain;
   wherein the binding domains are sufficiently complementary to two regions immediately flanking a purine:pyrimidine cleavage site within the c-Jun mRNA such that the DNAzyme cleaves the c-Jun mRNA.

As used herein, “DNAzyme” means a DNA molecule that specifically recognises and cleaves a distinct target nucleic acid sequence, which may be either DNA or RNA.

In a preferred embodiment, the binding domains of the DNAzyme are complementary to the regions immediately flanking the cleavage site. It will be appreciated by those skilled in the art, however, that strict complementarity may not be required for the DNAzyme to bind to and cleave the c-Jun mRNA.

The binding domain lengths (also referred to herein as “arm lengths”) can be of any permutation, and can be the same or different. In a preferred embodiment, the binding domain lengths are at least 6 nucleotides, and preferably 9 nucleotides. Preferably, both binding domains have a combined total length of at least 14 nucleotides. Various permutations in the length of the two binding domains, such as 7+7, 8+8 and 9+9, are envisioned. Preferably, the length of the two binding domains are 9+9.

The catalytic domain of a DNAzyme of the present invention may be any suitable catalytic domain. Examples of suitable catalytic domains are described in Santoro & Joyce (1997) Proc. Natl. Acad. Sci. USA 94: 4262-4266 and U.S. Pat. No. 5,807,718. In a preferred embodiment, the catalytic domain has the nucleotide sequence GGCTAGCTACAACGA (SEQ ID No:2).

It is preferred that the DNAzyme cleavage site is within the region of residues A²⁸⁷ to A¹⁵⁰¹, more preferably U¹²⁹⁶ to G¹⁴⁹⁷, of the c-Jun mRNA. It is particularly preferred that the cleavage site within the c-Jun mRNA is the GU site corresponding to nucleotides G¹³¹¹U¹³¹².

In a further preferred embodiment, the DNAzyme has the sequence

(Dz23; SEQ ID No: 3)
5′cgggaggaaGGCTAGCTACAACGAgaggcgttg-3′.

In applying DNAzyme based treatments, it is preferable that the DNAzymes be as stable as possible against degradation in the intra cellular milieu. One means of accomplishing this is by incorporating a 3′ 3′ inversion at one or more termini of the DNAzyme. More specifically, a 3′ 3′ inversion (also referred to herein simply as an “inversion”) means the covalent phosphate bonding between the 3′ carbons of the terminal nucleotide and its adjacent nucleotide. This type of bonding is opposed to the normal phosphate bonding between the 3′ and 5′ carbons of adjacent nucleotides, hence the term “inversion”. Accordingly, in a preferred embodiment, the 3′ end nucleotide residue is inverted in the building domain contiguous with the 3′ end of the catalytic domain. In addition to inversions, the instant DNAzymes may contain modified nucleotides. Modified nucleotides include, for example, N3′ P5′ phosphoramidate linkages, and peptide nucleic acid linkages. These are well known in the art.

In a particularly preferred embodiment, the DNAzyme includes an inverted T at the 3′ position.

In order to increase resistance to exonucleolytic degradation and helical thermostability locked nucleic acid analogues can be produced. Further information regarding these analogues is provided in Vester et al. (2002) J. Am. Chem. Soc. 124(46): 13682-13683, the disclosure of which is incorporated herein by reference.

In another embodiment, the method is achieved by inhibiting translation of the c-Jun mRNA using synthetic antisense DNA molecules that do not act as a substrate for RNase and act by sterically blocking gene expression.

In another embodiment, the method is achieved by inhibiting translation of the c-Jun mRNA by destabilising the mRNA using synthetic antisense DNA molecules that act by directing the RNase degradation of the c-Jun mRNA present in the heteroduplex formed between the antisense DNA and mRNA.

In one preferred embodiment of the present invention, the antisense oligonucleotide comprises a sequence which hybridises to c-Jun within the region of residues U¹²⁹⁶ to G¹⁴⁹⁷.

It will be understood that the antisense oligonucleotide need not hybridise to this whole region. It is preferred that the antisense oligonucleotide has the sequence

| | |
|---|---|
| CGGGAGGAACGAGGCGTTG. | (SEQ ID No: 4) |

In another embodiment, the method is achieved by inhibiting translation of the c-Jun mRNA by cleavage of the mRNA by sequence specific hammerhead ribozymes and derivatives of the hammerhead ribozyme such as the Minizymes or Mini ribozymes or where the ribozyme is derived from:
(i) the hairpin ribozyme,
(ii) the Tetrahymena Group I intron,
(iii) the Hepatitis Delta Viroid ribozyme or
(iv) the Neurospera ribozyme.

It will be appreciated by those skilled in the art that the composition of the ribozyme may be;
(i) made entirely of RNA,
(ii) made of RNA and DNA bases, or
(iii) made of RNA or DNA and modified bases, sugars and backbones

Within the context of the present invention, the ribozyme may also be either;
(i) entirely synthetic or
(ii) contained within a transcript from a gene delivered within a virus derived vector, expression plasmid, a synthetic gene, homologously or heterologously integrated into the patients genome or delivered into cells ex vivo, prior to reintroduction of the cells of the patient, using one of the above methods.

It is preferred that the ribozyme cleaves the c-Jun mRNA in the region of residues U¹²⁹⁶ to G¹⁴⁹⁷.

In another embodiment, the method is achieved by inhibition of the ability of the c-Jun gene to bind to its target DNA by expression of an antisense c-Jun mRNA.

In a still further embodiment the nucleic acid is dsRNA targeted against c-Jun mRNA, a nucleic acid molecule which results in production of dsRNA targeted against c-Jun mRNA or small interfering RNA (siRNA) molecules targeted against c-Jun mRNA. So called “RNA interference” or “RNAi” is well known and further information regarding RNAi is provided in Hannon (2002) Nature 418: 244-251, McManus & Sharp (2002) Nature Reviews: Genetics 3(10): 737-747, and Bhindi et al (2007) Am J Pathol, in press, the disclosures of which are incorporated herein by reference.

Small interfering RNA (siRNA), sometimes known as short interfering RNA or silencing RNA, are a class of 20-25 nucleotide-long double-stranded RNA molecules (comprising a sense strand and an antisense strand), that play a variety of roles in biology. Most notably, siRNA is involved in the RNA interference (RNAi) pathway where the siRNA interferes with the expression of a specific gene.

In a preferred embodiment of the present invention, the siRNA sense strand is selected from the group consisting of AAGUCAUGAACCACGUUAACA (SEQ ID No:5), AAGAACUGCAUGGACCUAACA (SEQ ID No:6), CAGCUUCAUGCCUUUGUAA (SEQ ID No:7), and CAGCUUCAUGCCUUUGUAA (SEQ ID No:13)

The present invention also contemplates chemical modification(s) of siRNAs that enhance siRNA stability and support their use in vivo (see for example, Shen et al. (2006) Gene Therapy 13: 225-234). These modifications might include inverted abasic moieties at the 5′ and 3′ end of the sense strand oligonucleotide, and a single phosphorothioate linkage between the last two nucleotides at the 3′ end of the antisense strand.

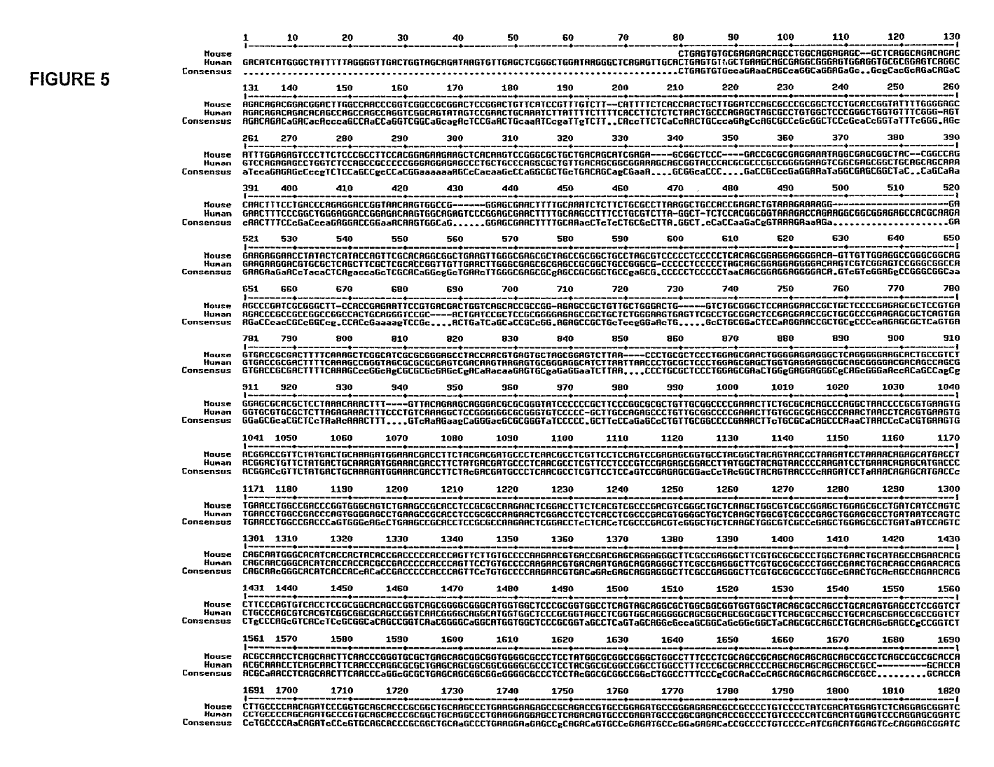

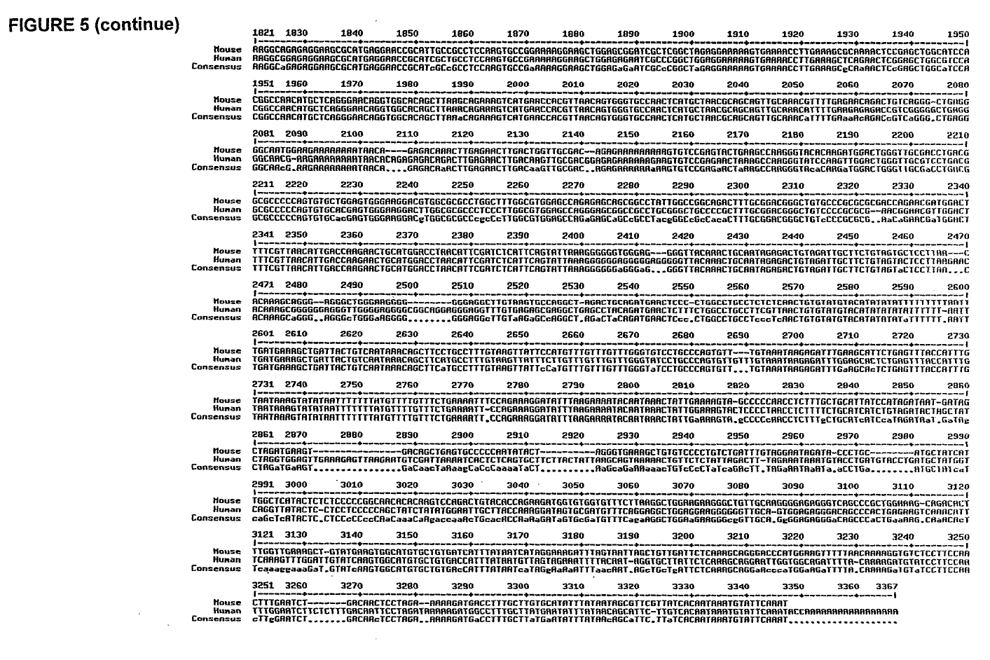

It will be appreciated by a person skilled in the art that, in the in vitro and in vivo experimental examples which follow, the nucleic acids which decrease the level of c-Jun mRNA, c-Jun mRNA translation or nuclear accumulation or activity of c-Jun protein are demonstrated in a murine model. The methods and compositions of present invention are intended for application in humans, and it will be further appreciated by a person skilled in the art that there are differences between murine c-Jun mRNA (SEQ ID No:8) and human c-Jun mRNA (SEQ ID No:9) sequences, differences which would be taken into account when selecting the inhibitory nucleic acid molecules of the invention. A sequence alignment of mouse and human c-Jun sequences is given in FIG. 5.

The murine c-Jun mRNA sequence (SEQ ID No:8) is as follows:

| | |
|---|---|
| cugagugugc gagagacagc cuggcaggag agcgcucagg cagacagaca gacagacgga | 60 |
| cggacuuggc caacccgguc ggccgcggac uccggacugu ucauccguuu gucuucauuu | 120 |
| ucucaccaac ugcuuggauc cagcgcccgc ggcuccugca ccgguauuuu ggggagcauu | 180 |
| uggagagucc cuucucccgc cuuccacgga gaagaagcuc acaaguccgg gcgcugcuga | 240 |
| cagcaucgag agcggcuccc gaccgcgcga ggaaauaggc gagcggcuac cggccagcaa | 300 |
| cuuuccugac ccagaggacc gguaacaagu ggccgggagc gaacuuuugc aaaucucuuc | 360 |
| ugcgccuuaa ggcugccacc gagacuguaa agaaaaggga gaagaggaac cuauacucau | 420 |
| accaguucgc acaggcggcu gaaguugggc gagcgcuagc cgcggcugcc uagcgucccc | 480 |
| cucccccuca cagcggagga ggggacaguu guuggaggcc gggcggcaga gcccgaucgc | 540 |
| gggcuuccac cgagaauucc gugacgacug gucagcaccg ccggagagcc gcuguugcug | 600 |
| ggacuggucu gcgggcucca aggaaccgcu gcuccccgag agcgcuccgu gagugaccgc | 660 |
| gacuuuucaa agcucggcau cgcgcgggag ccuaccaacg ugagugcuag cggagucuua | 720 |
| acccugcgcu cccuggagcg aacuggggag gagggcucag ggggaagcac ugccgucugg | 780 |
| agcgcacgcu ccuaaacaaa cuuuguuaca gaagcaggga cgcgcgggua uccccccgcu | 840 |
| ucccggcgcg cuguugcggc cccgaaacuu cugcgcacag cccaggcuaa ccccgcguga | 900 |
| agugacggac cguucuauga cugcaaagau ggaaacgacc uucuacgacg augcccucaa | 960 |
| cgccucguuc cuccaguccg agagcggugc cuacggcuac aguaacccua agauccuaaa | 1020 |
| acagagcaug accuugaacc uggccgaccc ggugggcagu cugaagccgc accuccgcgc | 1080 |
| caagaacucg gaccuucuca cgucgcccga cgucgggcug cucaagcugg cgucgccgga | 1140 |
| gcuggagcgc cugaucaucc aguccagcaa ugggcacauc accacuacac cgacccccac | 1200 |
| ccaguucuug ugccccaaga acgugaccga cgagcaggag ggcuucgccg agggcuucgu | 1260 |
| gcgcgcccug gcugaacugc auagccagaa cacgcuuccc agugucaccu ccgcggcaca | 1320 |
| gccggucagc ggggcgggca ugguggcucc cgcgguggcc ucaguagcag gcgcuggcgg | 1380 |
| cggugguggc uacagcgcca gccugcacag ugagccuccg gucuacgcca accucagcaa | 1440 |
| cuucaacccg ggugcgcuga gcagcggcgg uggggcgccc uccuauggcg cggccgggcu | 1500 |
| ggccuuuccc ucgcagccgc agcagcagca gcagccgccu cagccgccgc accacuugcc | 1560 |
| ccaacagauc ccggugcagc acccgcggcu gcaagcccug aaggaagagc cgcagaccgu | 1620 |
| gccggagaug ccgggagaga cgccgccccu guccccuauc gacauggagu cucaggagcg | 1680 |
| gaucaaggca gagaggaagc gcaugaggaa ccgcauugcc gccuccaagu gccggaaaag | 1740 |
| gaagcuggag cggaucgcuc ggcuagagga aaaagugaaa accuugaaag cgcaaaacuc | 1800 |
| cgagcuggca uccacggcca acaugcucag ggaacaggug gcacagcuua agcagaaagu | 1860 |
| caugaaccac guuaacagug ggugccaacu caugcuaacg cagcaguugc aaacguuuug | 1920 |
| agaacagacu gucagggcug aggggcaaug gaagaaaaaa aauaacagag acaaacuuga | 1980 |
| gaacuugacu gguugcgaca gagaaaaaaa aaguguccga guacugaagc caaggguaca | 2040 |
| caagauggac uggguugcga ccugacggcg cccccagugu gcuggagugg gaaggacgug | 2100 |
| gcgcgccugg cuuuggcgug gagccagaga gcagcggccu auuggccggc agacuuugcg | 2160 |
| gacgggcugu gcccgcgcgc gaccagaacg auggacuuuu cguuaacauu gaccaagaac | 2220 |
| ugcauggacc uaacauucga ucucauucag uauuaaaggg gggugggagg gguuacaaac | 2280 |
| ugcaauagag acuguagauu gcuucuguag ugcuccuuaa cacaaagcag ggagggcugg | 2340 |
| gaaggggggg aggcuuguaa gugccaggcu agacugcaga ugaacucccc uggccugccu | 2400 |
| cucucaacug uguauguaca uauauauuuu uuuuuaauuu gaugaaagcu gauuacuguc | 2460 |
| aauaaacagc uuccugccuu uguaaguuau uccauguuug uuuguuuggg uguccugccc | 2520 |
| aguguuugua aauaagagau uugaagcauu cugaguuuac cauuuguaau aaaguauaua | 2580 |
| auuuuuuuau guuuuguuuc ugaaaauuuc cagaaaggau auuuaagaaa auacaauaaa | 2640 |
| cuauugaaaa guagccccca accucuuugc ugcauuaucc auagauaaug auagcuagau | 2700 |
| gaagugacag cugagugccc ccaauauacu agggugaaag cugugucccc ugucugauuu | 2760 |
| guaggaauag auacccugca ugcuaucauu ggcucauacu cucucccccg gcaacacaca | 2820 |
| aguccagacu guacaccaga agauggugug guguuucuua aggcuggaag aagggcuguu | 2880 |
| gcaaggggag agggucagcc cgcuggaaag cagacacuuu gguugaaagc uguaugaagu | 2940 |
| ggcaugugcu gugaucauuu auaaucauag gaaagauuua guaauuagcu guugauucuc | 3000 |
| aaagcaggga cccauggaag uuuuuaacaa aaggugucuc cuuccaacuu ugaaucugac | 3060 |
| aacuccuaga aaaagaugac cuuugcuugu gcauauuuau aauagcguuc guuaucacaa | 3120 |
| uaaauguauu caaau | 3135 |

Similarly, the human c-Jun mRNA sequence (SEQ ID No: 9) is as follows:

| | |
|---|---|
| gacaucaugg gcuauuuuua gggguugacu gguagcagau aaguguugag cucgggcugg | 60 |
| auaagggcuc agaguugcac ugaguguggc ugaagcagcg aggcgggagu ggaggugcgc | 120 |
| ggagucaggc agacagacag acacagccag ccagccaggu cggcaguaua guccgaacug | 180 |
| caaaucuuau uuucuuuuca ccuucucucu aacugcccag agcuagcgcc uguggcuccc | 240 |
| gggcuggugu uucgggagug uccagagagc cuggucucca gccgcccccg ggaggagagc | 300 |
| ccugcugccc aggcgcuguu gacagcggcg gaaagcagcg guacccacgc gcccgccggg | 360 |
| ggaagucggc gagcggcugc agcagcaaag aacuuucccg gcugggagga ccggagacaa | 420 |
| guggcagagu cccggagcga acuuuugcaa gccuuuccug cgucuuaggc uucuccacgg | 480 |
| cgguaaagac cagaaggcgg cggagagcca cgcaagagaa gaaggacgug cgcucagcuu | 540 |
| cgcucgcacc gguuguugaa cuugggcgag cgcgagccgc ggcugccggg cgcccccucc | 600 |
| cccuagcagc ggaggagggg acaagucguc ggaguccggg cggccaagac ccgccgccgg | 660 |
| ccggccacug caggguccgc acugauccgc uccgcgggga gagccgcugc ucugggaagu | 720 |
| gaguucgccu gcggacuccg aggaaccgcu gcgcccgaag agcgcucagu gagugaccgc | 780 |
| gacuuuucaa agccggguag cgcgcgcgag ucgacaagua agagugcggg aggcaucuua | 840 |
| auuaacccug cgcucccugg agcgagcugg ugaggagggc gcagcgggga cgacagccag | 900 |
| cgggugcgug cgcucuuaga gaaacuuucc cugucaaagg cuccgggggg cgcggguguc | 960 |
| ccccgcuugc cagagcccug uugcggcccc gaaacuugug cgcgcagccc aaacuaaccu | 1020 |
| cacgugaagu gacggacugu ucuaugacug caaagaugga aacgaccuuc uaugacgaug | 1080 |
| cccucaacgc cucguuccuc ccguccgaga gcggaccuua uggcuacagu aaccccaaga | 1140 |
| uccugaaaca gagcaugacc cugaaccugg ccgacccagu ggggagccug aagccgcacc | 1200 |
| uccgcgccaa gaacucggac cuccucaccu cgcccgacgu ggggcugcuc aagcuggcgu | 1260 |
| cgcccgagcu ggagcgccug auaauccagu ccagcaacgg gcacaucacc accacgccga | 1320 |
| cccccaccca guuccugugc cccaagaacg ugacagauga gcaggagggc uucgccgagg | 1380 |
| gcuucgugcg cgcccuggcc gaacugcaca gccagaacac gcugcccagc gucacgucgg | 1440 |
| cggcgcagcc ggucaacggg gcaggcaugg uggcucccgc gguagccucg guggcagggg | 1500 |
| gcagcggcag cggcggcuuc agcgccagcc ugcacagcga gccgccgguc uacgcaaacc | 1560 |
| ucagcaacuu caacccaggc gcgcugagca gcggcggcgg ggcgcccucc uacggcgcgg | 1620 |
| ccggccuggc cuuucccgcg caaccccagc agcagcagca gccgccgcac caccugcccc | 1680 |
| agcagaugcc cgugcagcac ccgcggcugc aggcccugaa ggaggagccu cagacagugc | 1740 |
| ccgagaugcc cggcgagaca ccgccccugu cccccaucga cauggagucc caggagcgga | 1800 |
| ucaaggcgga gaggaagcgc augaggaacc gcaucgcugc cuccaagugc cgaaaaagga | 1860 |
| agcuggagag aaucgcccgg cuggaggaaa aagugaaaac cuugaaagcu cagaacucgg | 1920 |
| agcuggcguc cacggccaac augcucaggg aacagguggc acagcuuaaa cagaaaguca | I980 |
| ugaaccacgu uaacaguggg ugccaacuca ugcuaacgca gcaguugcaa acauuuugaa | 2040 |
| gagagaccgu cgggggcuga ggggcaacga agaaaaaaaa uaacacagag agacagacuu | 2100 |
| gagaacuuga caaguugcga cggagagaaa aaagaagugu ccgagaacua aagccaaggg | 2160 |
| uauccaaguu ggacuggguu gcguccugac ggcgccccca gugugcacga gugggaagga | 2220 |
| cuuggcgcgc ccucccuugg cguggagcca gggagcggcc gccugcgggc ugccccgcuu | 2280 |
| ugcggacggg cuguccccgc gcgaacggaa cguuggacuu uucguuaaca uugaccaaga | 2340 |
| acugcaugga ccuaacauuc gaucucauuc aguauuaaag gggggagggg gaggggguua | 2400 |
| caaacugcaa uagagacugu agauugcuuc uguaguacuc cuuaagaaca caaagcgggg | 2460 |
| ggaggguugg ggaggggcgg caggagggag guuugugaga gcgaggcuga gccuacagau | 2520 |
| gaacucuuuc uggccugccu ucguuaacug uguauguaca uauauauauu uuuuaauuug | 2580 |
| augaaagcug auuacuguca auaaacagcu ucaugccuuu guaaguuauu ucuuguuugu | 2640 |
| uuguuugggu auccagccca guguuguuug uaaauaagag auuuggagca cucugaguuu | 2700 |
| accauuugua auaaaguaua uaauuuuuuu auguuuuguu ucugaaaauu ccagaaagga | 2760 |
| uauuuaagaa aauacaauaa acuauuggaa aguacucccc uaaccucuuu ucugcaucau | 2820 |
| cuguagauac uagcuaucua gguggaguug aaagaguuaa gaaugucgau uaaaaucacu | 2880 |
| cucagugcuu cuuacuauua agcaguaaaa acuguucucu auuagacuuu agaaauaaau | 2940 |
| guaccugaug uaccugaugc uauggucagg uuauacuccu ccucccccag cuaucuauau | 3000 |
| ggaauugcuu accaaaggau agugcgaugu uucaggaggc uggaggaagg gggguugcag | 3060 |
| uggagaggga cagcccacug agaagucaaa cauuucaaag uuuggauugu aucaaguggc | 3120 |
| augugcugug accauuuaua auguuaguag aaauuuuaca auaggugcuu auucucaaag | 3180 |
| caggaauugg uggcagauuu uacaaaagau cuauccuucc aauuuggaau cuucucuuug | 3240 |
| acaauuccua gauaaaaaga uggccuuugc uuaugaauau uuauaacagc auucuuguca | 3300 |
| caauaaaugu auucaaauac caaaaaaaaa aaaaaaaa | 3338 |

Accordingly, in a preferred embodiment of the present invention, the DNAzyme targeted against c-Jun, a c-Jun antisense oligonucleotide, a ribozyme targeted against c-Jun, or ssDNA targeted against c-Jun dsDNA such that the ssDNA forms a triplex with the c-Jun dsDNA cleaves human c-Jun mRNA (SEQ ID No:9). In an alternative embodiment of the present invention, the dsRNA targeted against c-Jun mRNA, a nucleic acid molecule which results in production of dsRNA targeted against c-Jun mRNA or small interfering RNA molecules (siRNA) targeted against c-Jun mRNA cleaves human c-Jun mRNA (SEQ ID No:9).

In another embodiment, the method of the present invention is achieved by targeting the c-Jun gene directly using triple helix (triplex) methods in which a ssDNA molecule can bind to the dsDNA and prevent transcription.

In another embodiment, the method is achieved by inhibiting transcription of the c-Jun gene using nucleic acid transcriptional decoys. Linear sequences can be designed that form a partial intramolecular duplex which encodes a binding site for a defined transcriptional factor.

In another embodiment, the method is achieved by inhibition of c-Jun activity as a transcription factor using transcriptional decoy methods.

In another embodiment, the method is achieved by inhibition of the ability of the c-Jun gene to bind to its target DNA by drugs that have preference for GC rich sequences. Such drugs include nogalamycin, hedamycin and chromomycin A329.

In a second aspect of the present invention there is provided a pharmaceutical composition comprising a therapeutically effective amount of a nucleic acid which decreases the level of c-Jun mRNA, c-Jun mRNA translation or nuclear accumulation or activity of c-Jun protein, together with a pharmaceutically excipient, for treating or inhibiting arthritis.

Administration of the inhibitory nucleic acid may be effected or performed using any of the various methods and delivery systems known to those skilled in the art. The administering can be performed, for example, intra-articularly, intravenously, orally, via implant, transmucosally, transdermally, topically, intramuscularly, subcutaneously or extracorporeally. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition. In one embodiment the delivery vehicle contains Mg²⁺ or other cation(s) to serve as co factor(s) for efficient DNAzyme bioactivity.

In a preferred embodiment of the present invention, the nucleic acid molecule is administered by intra-articular injection. Local administration, such as via the intra-articular route, is a means of achieving high drug concentrations at a target site while reducing the likelihood of systemic inadvertent side effects.

Injectable drug delivery systems include solutions, suspensions, gels, microspheres and polymeric injectables, and can comprise excipients such as solubility altering agents (e.g., ethanol, propylene glycol and sucrose) and polymers (e.g., polycaprylactones and PLGA's). Implantable systems include rods and discs, and can contain excipients such as PLGA and polycaprylactone.

Transdermal delivery systems include patches, gels, tapes and creams, and can contain excipients such as solubilizers, permeation enhancers (e.g., fatty acids, fatty acid esters, fatty alcohols and amino acids), hydrophilic polymers (e.g., polycarbophil and polyvinylpyrolidone), and adhesives and tackifiers (e.g., polyisobutylenes, silicone based adhesives, acrylates and polybutene).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti caking agents, coating agents, and chelating agents (e.g., EDTA).

Topical delivery systems include, for example, gels and solutions, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In the preferred embodiment, the pharmaceutically acceptable carrier is a liposome or a biodegradable polymer. Examples of carriers which can be used in this invention include the following: (1) Fugene6® (Roche); (2) SUPERFECT® (Qiagen); (3) Lipofectamine 2000® (GIBCO BRL); (4) CellFectin, 1:1.5 (M/M) liposome formulation of the cationic lipid N,NI,NII,NIII tetramethyl N,NI,NII,NIII tetrapalmitylspermine and dioleoyl phosphatidyl ethanolamine (DOPE) (GIBCO BRL); (5) Cytofectin GSV, 2:1 (M/M) liposome formulation of a cationic lipid and DOPE (Glen Research); (6) DOTAP (N[1 (2,3 dioleoyloxy)N,N,N trimethyl ammoniummethylsulfate) (Boehringer Mannheim, Avanti Polar Lipids); (7) DODAP (Avanti Polar Lipids); and (8) Lipofectamine, 3:1 (M/M) liposome formulation of the polycationic lipid DOSPA and the neutral lipid DOPE (GIBCO BRL).

Delivery of the nucleic acids described may also be achieved via one or more of the following non limiting examples of vehicles:
(a) liposomes and liposome protein conjugates and mixtures;
(b) non liposomal lipid and cationic lipid formulations;
(c) activated dendrimer formulations;
(d) within a polymer formulation such as polyethylenimine (PEI) or pluronic gels or within ethylene vinyl acetate copolymer (EVAc). The polymer is preferably delivered intra luminally;
(e) within a viral liposome complex, such as Sendai virus; or
(f) as a peptide DNA conjugate.

Determining the therapeutically effective dose of the instant pharmaceutical composition can be done based on animal data using routine computational methods. In one embodiment, the therapeutically effective does contains between about 0.1 mg and about 1 g of the instant DNAzyme. In another embodiment, the therapeutically effective dose contains between about 1 mg and about 100 mg of the instant DNAzyme. In a further embodiment, the therapeutically effective does contains between about 10 mg and about 50 mg of the instant DNAzyme. In yet a further embodiment, the therapeutically effective does contains about 25 mg of the instant DNAzyme.

In order that the nature of the present invention may be more clearly understood, preferred forms thereof will now be described with reference to the following non-limiting examples.

### Example 1

### Methods & Materials

### Murine Model of Proliferative Retinopathy

Postnatal day 6 (P6) C57BL/6 mice were exposed to hyperoxia (75% oxygen) for 4 days in Quantum-Air Maxi-Sealed cages (Hereford, UK). Following hyperoxic exposure, P10 mice were returned to normoxia, anaesthetised (17 mg/kg ketamine and 2.5 mg/kg xylazine) and a bolus intravitreal injection of 20 pg of the DNAzyme Dz13, 5′-CGGGAGGAAGGCTAGCTACAACGAGAGGCGTTG (3′-3′ T)-3′ (SEQ ID No:10); Dz13scr, 5′-GCGACGTGAGGCTAGCTACAACGAGTGGAGGAG (3′-3′ T)-3′ (SEQ ID No:11) or c-Jun siRNA, 5′-r(CAGCUUCCUGCCUUUGUAA)d(TT)-3′ (SEQ ID No:13); c-Jun siRNAscr, 5′-r(GAUUACUAGCCGUCUUCCU)d(TT)-3′ (SEQ ID No:12) in 2 μl saline containing 0.2 μl FuGENE6 (n=6-12 eyes per group) was administered using a 26 gauge bevelled needle attached to a micro-volume syringe (SGE International, Melbourne, Australia). The mice were left at room oxygen for a further 7 days before P17 pup eyes were enucleated and fixed in 10% formalin in PBS. Serial 6 μm cross-sections of whole eyes were cut sagitally, parallel to the optic nerve, and stained with H&E. Blood vessels from each group were quantitated under light microscopy and expressed as the mean±SEM.

### Passive Cutaneous Anaphylaxis

Female six week-old Balb/c mice were injected with 25 ng mouse monoclonal anti-dinitrophenyl (DNP) IgE (Sigma) in PBS, pH 7.4 in one ear or with PBS in the other ear. Where indicated, mouse anti-DNP IgE in saline was co-administered with 100 μg DNAzyme (Dz13 or Dz13scr; Tri-Link, synthesized with 3′-3′ linked inverted T) or scrambled DNAzyme in 25 it of vehicle (FuGENE6 (Roche Diagnostics) in PBS (3:20 vol:vol) containing 1 mM MgCl₂) in one ear and the same volume of vehicle in the other ear. After 20 h, mice were injected intravenously with 100 μl PBS containing 100 μg DNP-human serum albumin and 1% Evans blue dye (Sigma). Mice were euthanased 30 min later and a 6 mm disk biopsy of the ear was obtained with the injection site as the epicentre. Each disc was incubated in 200 μl 10% formamide at 55° C. for 6 h. Dye extravasation was quantitated at 610 nm, blanked with formamide. Values were corrected for background absorbance using an untreated patch of skin of identical size.

### Miles Assay

Anaesthetised six-week-old female nude Balb/c mice (17 mg/kg ketamine and 2.5 mg/kg xylazine) were injected with 150 μl 1% Evans blue solution into the tail vein. After 5 min, DNAzyme or scrambled DNAzyme in 20 μl vehicle or vehicle alone, was delivered into the mid dorsum by intradermal injection. After 1 h, 50 ng VEGF₁₆₅ (Sigma) in 20 μl PBS was injected into an adjacent location 1 mm away. Extravasation of Evans blue was determined after 90 min by carefully excising the skin around the injection site, incubating in 200 μl 10% formamide for 24 h at 55° C. and measuring optical density at 610 nm. As a negative control, 50 ng of BSA in 20 μl PBS was used. Absorbance at 610 nm was measured as described above.

DNAzyme Extraction from Injected Skin and Assessment of Cleavage Activity

Anaesthetised female 6 w.o Balb/c nude mice were injected intradermally into the mid-dorsum with 100 μg of Dz13. Skin was excised around the injection site after 5 and 60 min and placed in Lysing Matrix D homogenizing tubes (Q-BIOgene, Carlsbad Calif.) containing 1.2 ml TRIzol (Invitrogen, Carlsbad Calif.). Tissue was homogenized in a Fast Prep FP120 Bio 101 (Thermo Savant, Halbrook N.Y.) for 3 cycles at 20 sec/cycle. DNA was extracted according to the TRIzol protocol for DNA isolation and purified using P30 micro bio-spin columns (Bio Rad, Hercules Calif.). Synthetic RNA substrate (0.5 μg) was ³²P-labeled using T4 polynucleotide kinase and purified from unincorporated nucleotides using P30 micro bio spin columns. 2 μl of DNA isolated from tissue was incubated with 1 μl of labeled RNA substrate for 1 h at 37° C. 2 μl of the cleavage reaction was added to 4 ml of formamide loading dye and loaded onto a 12% denaturing PAGE gel. Cleavage products were visualized by autoradiography.

### Endothelial-Monocytic Cell Adhesion Assay

Human microvascular endothelial cells (HMEC-1) grown in 24-well plates at 80-90% confluence were transfected with 0.05 μM of the DNAzyme or siRNA (using FuGENE6) after changing the growth medium from 10% serum to serum-free. After 18 h, the cells were washed with PBS and fresh serum-free medium containing 20 ng/ml of IL-1beta was added. After 12 h, THP-1 monocytic cells were added to each well at density of 2.5×10⁵ cells per well. Alternatively, the THP-1 cells were transfected with 0.05 μM of Dz13 or Dz13scr and, after 18 h added to cytokine-treated endothelial cultures in 24-well plates at a density of 2.5×10⁵ cells per well. After 30 min, the wells were washed thrice with PBS to remove non-adherent cells. Monocytic cells adherent to endothelium were counted as the number of translucent cells per visual field using the 100× objective of a phase-contrast Olympus microscope.

### Rat Peritoneal Mesenteric Venule Inflammation

Male Sprague-Dawley rats (230-300 g) were anaesthetized with sodium thiobutabarbital (Inactin, 100 mg/kg injected intraperitoneally) and a tracheostomy performed for airway management throughout the experiment. A catheter was inserted into the right femoral artery for intravenous saline administration and blood pressure monitoring. Following midline abdominal incision, part of the mesentery from the small bowel was exteriorised and placed on a temperature-controlled Plexiglass chamber for observation of the mesenteric microcirculation using intravital microscopy. The small bowel and mesentery were continuously superfused with modified Krebs-Henseleit solution at 37° C. Mesenteric venules of 25-50 μm diameter and >100 μm length were selected. Images from an Olympus microscope were projected by a high-resolution colour video camera (JVC) into a colour high-resolution video-monitor and recorded on Super-VHS tapes. All images were analysed offline for 3 parameters of inflammation: leukocyte flux, adhesion and extravasation. Rolling leukocyte flux were measured by counting cells rolling past a defined reference point within the 100 μm vessel length per min. Leukocyte adhesion was assessed by counting leukocytes that remained stationary for at least 30 sec per 100 μm of length of vessel. Leukocyte numbers in tissue adjacent to the venule per microscopic field were used to quantitate extravasation. Venules were monitored for baseline flux, adhesion and extravasation 20-30 min prior to the commencement of each treatment. 100 μL of either vehicle or vehicle containing DNAzyme (35 μg) was applied topically and left undisturbed for 10 min during which time superfusion was temporarily stopped to facilitate DNAzyme infusion. Superfusion was resumed with either modified Krebs-Henseleit buffer or buffer containing IL-1beta (20 ng/ml). Video recordings for each treatment were made at the time of application of vehicle or vehicle plus DNAzyme and 60 min after application. The following exclusion criteria were used prior to addition of vehicle or IL-1: leukocyte flux >35 cells/min; >3 adherent cells per 100 μm of vessel; >10 extravasated leukocytes in the field of view after 20 min of undisturbed superfusion. Leukocyte flux, adhesion and extravasation were quantitated off-line at the conclusion of the experiment.

### Western Blot and Immunohistochemical Analysis

Western blot analysis was performed essentially as previously described using commercial rabbit or goat antipeptide polyclonal antibodies to c-Jun, E-selectin, VCAM-1, ICAM-1, VE-cadherin, JAM-1, PECAM-1, p-JNK-1 and beta-actin (Santa Cruz Biotechnology, Inc., R&D Systems, Alexis Biochemicals). Immunostaining was performed on formalin-fixed, paraffin-embedded mesenteric tissue with rabbit or goat polyclonal antipeptide antibodies essentially as described in Zhang et al. (2004) Journal of National Cancer Institute 96, 683-696.

### LPS-Induced Pulmonary Infiltration

DNAzyme (100 μg or 200 μg/50 μl) was administered into the lung via the nares of 7-8 week old Balb/c mice 2 h prior to LPS (Difco, E. coli) delivery (10 μg/40 μl). Control mice received 40 μl vehicle. Four hours after LPS administration, mice were sacrificed with an overdose of ketamine and xylazine (500 mg/kg and 50 mg/kg, respectively). Lungs were perfused by cardiac puncture via the right ventricle with saline then a tracheostomy performed with an 18-gauge needle. Bronchoalveolar lavage fluid was obtained by washing the lungs 3 times with 1 ml Hank's balanced salts solution. Cells were pelleted at 400 g for 5 min then resuspended in 200 μl of PBS. Neutrophils were counted using a hemocytometer and expressed as cell counts/μl.

### Collagen Antibody-Induced Arthritis

Arthritis was induced in 6-week old Balb/c mice by injection i.p. of a commercially-obtained (Chemicon International, USA) cocktail of 4 monoclonal antibodies to type II collagen (2 mg/mouse) followed by a second injection i.p. 72 h later of 50 μg LPS (Kagari et al. (2002) J Immunol 169, 1459-1466). DNAzyme (50 μg/5 μl) was administered directly (intraarticular route) into hind paw joint at the time of the second injection. After 9 days, the mice were sacrificed by cervical dislocation and hind paw thickness was determined using electronic Vernier callipers. Hind limbs were fixed in 10% formalin in PBS, decalcified in 30% formic acid and 10% formaldehyde in water for 24 h, their heels removed and processed into paraffin. 4-7 μm-thick sagittal sections across the heel were stained with standard H&E. The degree of inflammation in the synovial lining was evaluated by analysing the mean density of three randomly selected (using MS Excel) areas (0.1 mm²) in the medial aspect of the tibitarsal joint under 200× magnification using an Olympus BX60 microscope and a modified version of NIH Image software (ImageJ software, Wright Cell Imaging Facility, Toronto Western Research Institute). The relative proportions of polymorphonuclear and mononuclear cells in each section was evaluated by counting 3×100 cells in two to three adjacent high power fields (400× magnification) at the bone-synovial junction. To grade bone erosion paw sections were evaluated using a modified semi-quantitative scoring criteria previously described Bolon et al. (2004) Vet Pathol 41, 30-36). In brief, bone erosion score 0 represents normal bone integrity; 1: Minimal loss of cortical or trabecular bone; 2: Moderate loss of bone at the edges of talus and minimum loss in cortex of distal tibia; 3: Marked loss of bone the edges of talus and moderate loss in the cortex of distal tibia; 4: Marked loss of bone in both talus and tibia. For consistency, scoring was performed on the talus and tibia under 200× magnification.

### DNAzyme Localization Studies

20 μg of FITC-DNAzyme (TriLink-BioTechnologies, San Diego USA) was injected intraarticularly (CAIA model), intradermally (Miles or PCA assay) or intravitreally (ROP model) into anaesthetized (17 mg/kg ketamine, 2.5 mg/kg xylazine) female 6 w.o. Balb/c, Balb/C nude or C57BL/6 mice respectively. In the lung model, 20 μg of the FITC-DNAzyme was delivered by inhalation to female 6 w.o. Balb/c mice. Areas of tissue localization were removed from over-anaesthetized mice (100 mg/kg ketamine, 5 mg/kg xylazine) and visualized by fluoroscopy at 400× magnification.

### Animal Ethics and Statistical Analysis

All animal experiments were approved by the Animal Care and Ethics Committee, The University of New South Wales, and purchased from the Biological Resources Centre, University of New South Wales. All values are expressed as the mean±S.E.M. Differences between groups were tested for statistical significance using Student's t-test or analysis of variance (ANOVA). Differences were considered to be significant at P<0.05.

### Example 2

### Results

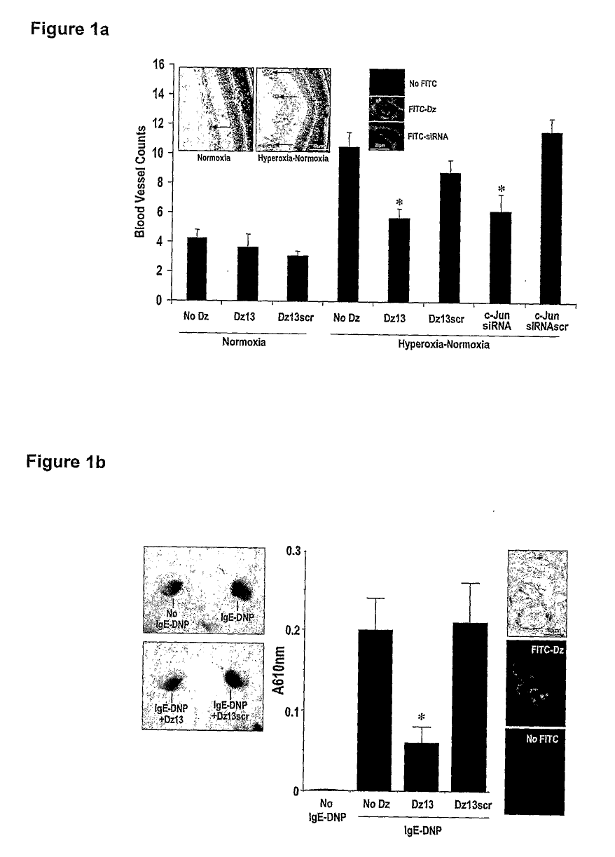

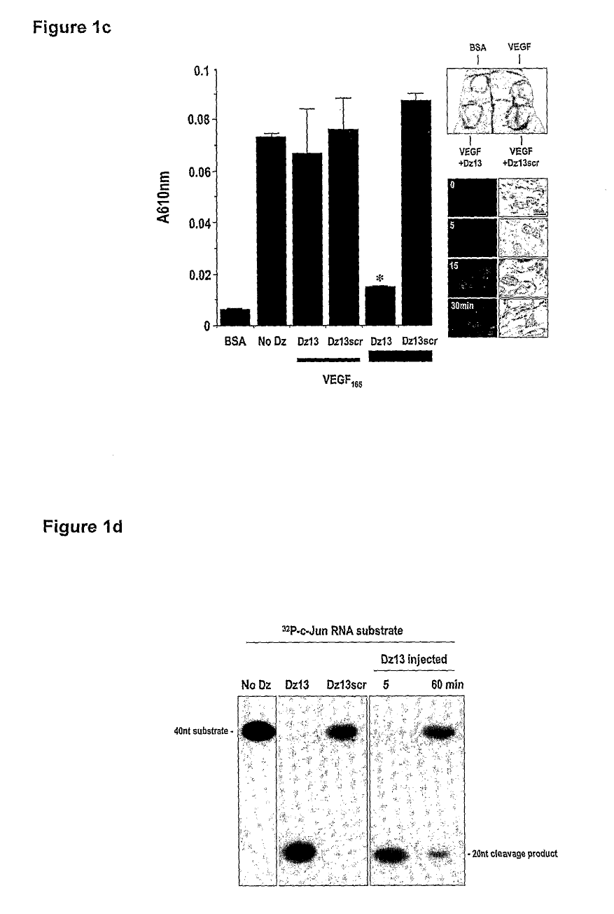

Exposure of neonatal mice to hyperoxic conditions followed by normoxia results in retinal neovascularization (Smith et al. (1994) Invest Ophthalmol Vis Sci 35, 101-111; FIG. 1a). Single intravitreal administration of Dz13 (20 μg) significantly inhibited retinal neovascularization compared to mice treated with an identical amount of the Dz13scr, in which the catalytic domain of Dz13 is retained but the hybridising arms of Dz13 are scrambled (FIG. 1a). Retinal neovascularization was also inhibited following intravitreal delivery of synthetic siRNA targeting c-Jun, but not by its sequence-scrambled counterpart, siRNAscr (FIG. 1a). The Dz13 and the siRNA target sequences in murine c-Jun mRNA (NM_{—}010591) are separated by approximately 1.5 kb (Dz13 targets nts 958-976; cleavage at G⁹⁶⁷) whereas the siRNA is directed at nts 2465-2485). Fluorescence microscopy following administration of the DNAzyme or siRNA bearing fluorescein isothiocyanate (FITC) moieties confirmed delivery to the vascular endothelial lining (FIG. 1a). No fluorescent signal was detected with either nucleic acid molecule not conjugated with FITC (FIG. 1a) thereby excluding artefact caused by autofluorescence.

The inventors determined whether Dz13 could influence vascular permeability using passive cutaneous anaphylaxis in mice. Vascular leakage in this model is detected by Evans blue dye extravasation from the bloodstream into tissue as a consequence of IgE-DNP/DNP-induced passive cutaneous anaphylaxis (FIG. 1b, upper left panel). Local injection of a single dose (100 μg) of Dz13 was sufficient to inhibit the vascular response in the ears of Balb/c mice by 70% (FIG. 1b, lower left and middle panels). In contrast, Dz13scr had no inhibitory effect (FIG. 1b, lower left and middle panels). Experiments using FITC-labeled DNAzyme demonstrated localization in endothelium (FIG. 1b, right panels).

The inventors further investigated the capacity of Dz13 to inhibit vascular permeability using the Miles assay in athymic Balb/c nude mice. In this model, the intradermal administration of VEGF₁₆₅ causes leakage of Evans blue dye from the circulation into tissue. Intradermal injection of VEGF₁₆₅ induced dye leakage within 90 min (FIG. 1c). This was blocked 80% by prior local administration of a single dose (100 μg) of Dz13, but not Dz13scr (FIG. 1c). In contrast, 10 μg of Dz13 in the same volume of vehicle had no effect on dye leakage (FIG. 1c) indicating therefore that Dz13 inhibition of vascular permeability is dose-dependent. FITC-labeled DNAzyme localized to the endothelium and surrounding structures in a time-dependent manner (FIG. 1c, lower right panels). DNAzyme Dz14 (Khachigian et al. (2002) J. Biol. Chem. 277, 22985-22991), which targets nucleotides 1145-1162 (cleavage at A¹¹⁵⁴) in murine c-Jun mRNA (Dz13 and Dz14, incidently target G¹³¹¹ and A¹⁴⁹⁸ in human c-Jun mRNA, respectively) did not affect dye extravasation in this model (data not shown) consistent with our previous demonstration that Dz14 does not cleave c-Jun mRNA nor influence cell proliferation (Khachigian et al. (2002) J. Biol. Chem. 277, 22985-22991). To demonstrate that Dz13 retained its activity after intradermal injection, DNA was extracted from the skin at various times then added to a standard in vitro cleavage reaction with ³²P-labeled 40 nt synthetic RNA substrate. FIG. 1d demonstrates that Dz13 was catalytically-active 5 min after delivery. Cleavage product was apparent even after 60 min (FIG. 1d) albeit less product formed, the likely result of distal tissue distribution over time. The 3′-3′-linked inverted thymidine in DNAzyme confers improved stability against nucleolytic degradation (Santiago et al. (1999) Nature Med. 5, 1264-1269).

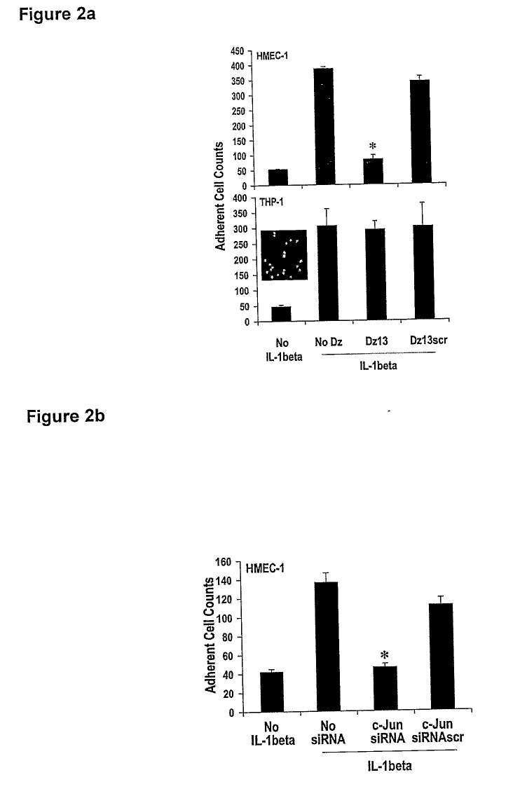

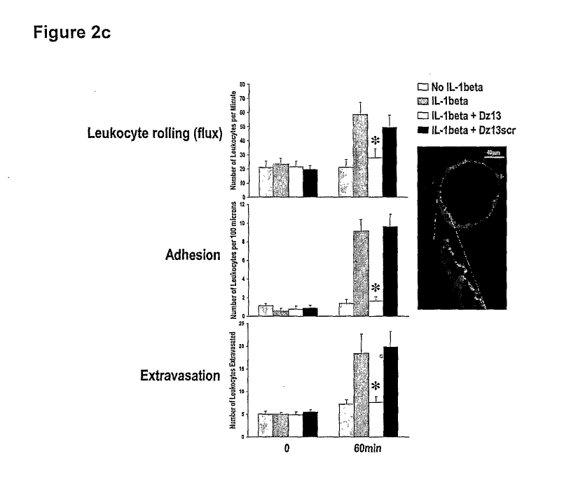

The preceding data showing Dz13 inhibition of vascular leakiness led us to investigate whether c-Jun also played a role in leukocyte infiltration through permeable endothelium. First, using an in vitro co-culture model, the inventors determined whether c-Jun was required for monocytic cell adhesion. IL-1beta stimulated THP-1 monocytic cell adhesion to human microvascular endothelial cell (HMEC-1 line) monolayers by 6-7-fold within 30 min (FIG. 2a). Prior transfection of endothelial cells with Dz13, unlike Dz13scr, virtually abolished monocytic cell-endothelial adhesion (FIG. 2a, upper panel). Similar results were obtained using c-Jun siRNA, but not scrambled siRNA (FIG. 2b). In contrast, Dz13 failed to inhibit cytokine-inducible adhesion when the monocytic cells were transfected (FIG. 2a, lower panel) despite DNAzyme incorporation in virtually the entire population (FIG. 2a, lower panel inset). These findings indicate that Dz13 inhibition of monocytic cell adhesion to cytokine-challenged endothelium relies upon endothelial rather than monocytic cell transfection of DNAzyme.

The inventors next investigated the capacity of Dz13 to inhibit inflammation in the rat mesenteric microcirculation. IL-1beta induced leukocyte flux (FIG. 2c, upper panel), adhesion (FIG. 2c, middle panel) and extravasation (FIG. 2c, lower panel) in mesenteric venules within 60 min of superfusion. All three processes were completely abrogated by topical delivery of a single dose (35 μg) of Dz13 for 10 min prior to cytokine exposure, whereas the same amount of Dz13scr had no effect (FIG. 2c). Fluorescence microscopy on cross-sections of mesenteric venules pre-treated with FITC-labeled DNAzyme prior to IL-1beta administration confirmed DNAzyme uptake into venular endothelium (FIG. 2c, right panel).

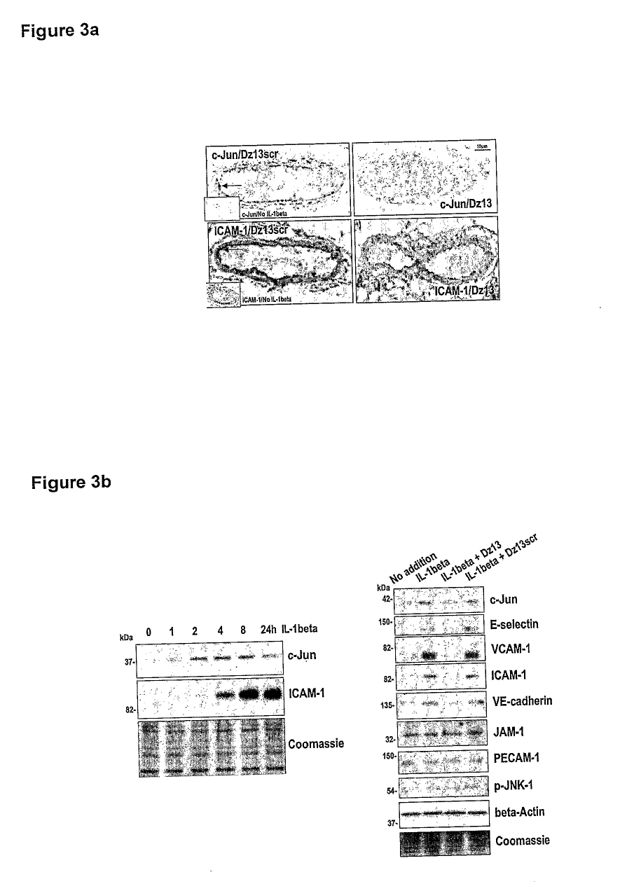

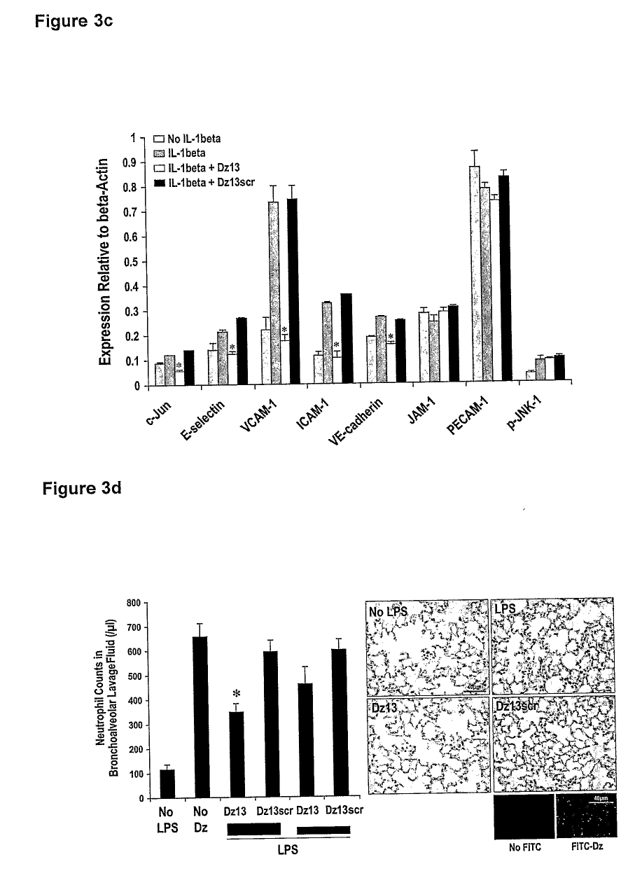

The multi-staged process of leukocyte trafficking through endothelium is mediated, at the molecular level, by the dynamic regulation of genes whose products mediate leukocyte rolling, adhesion and extravasation. These genes are in turn regulated by transcription factors whose expression is exquisitely sensitive to changes in the local humoral milieu. To gain insight into the genes regulated by c-Jun in this process, the inventors performed serial immunohistochemical analysis on DNAzyme-treated mesenteric tissue. Dz13, but not Dz13scr, inhibited c-Jun, E-selectin, vascular cell adhesion molecule (VCAM-1), intercellular adhesion molecule-1 (ICAM-1) and VE-cadherin expression in venule endothelium (FIG. 3a and Table 1), whereas junctional adhesion molecule-1 (JAM-1), platelet-endothelial cell adhesion molecule-1 (PECAM-1) and c-Fos levels were unaffected (FIG. 3a and Table 1). E-selectin mediates leukocyte rolling across activated endothelium, VCAM-1 and ICAM-1 facilitate leukocyte engagement, whereas the junctional molecules PECAM-1, VE-cadherin and JAM-1 regulate vascular permeability and leukocyte trans-endothelial migration (Engelhardt & Wolburg (2004) Eur. J. Immunol. 34, 2955-2963). Dz13 therefore suppressed the expression of molecules involved in all stages of the inflammatory process. E-selectin (Min & Pober (1997) J Immunol 159, 3508-3518), VCAM-1 (Ahmad et al. (1998) J Biol Chem 273, 4616-4621) and ICAM-1 (Wang et al. (1999) Arterioscler Thromb Vasc Biol. 19, 2078-2084) are c-Jun-dependent genes. Although whether c-Jun directly regulates VE-cadherin transcription is not presently known, the rodent VE-cadherin promoter contains c-Jun recognition elements.

TABLE 1

| Antigen | Dz13 | Dz13scr |
|---|---|---|
| c-Jun | − | ++ |
| E-selectin | − | ++ |
| VCAM-1 | − | ++ |
| ICAM-1 | − | +++ |
| VE-cadherin | − | ++ |
| JAM-1 | ++ | ++ |
| PECAM-1 | ++ | ++ |
| c-Fos | ++/+++ | ++/+++ |

Blinded score scale: − = no staining; +/− = occasional; + = weak; ++ = moderate; +++ = intense immunostaining.

Western blot analysis revealed that IL-1beta stimulates c-Jun expression in microvascular endothelial cells in a time-dependent manner (FIG. 3b, left panel). The inducible expression of c-Jun within 1 h preceded that of ICAM-1, which was not apparent until after 2 h (FIG. 3b, left panel). Dz13 inhibited IL-1beta-inducible c-Jun expression (FIG. 3b, right panel and FIG. 3c), whereas Dz13scr had no effect (FIG. 3b, right panel and FIG. 3c). The DNAzyme also inhibited cytokine-inducible E-selectin, VCAM-1, ICAM-1 and VE-cadherin expression (FIG. 3b, right panel and FIG. 3c), but did not affect levels of JAM-1 or PECAM-1 (FIG. 3b, right panel and FIG. 3c), nor did it influence the phosphorylation of c-Jun N-terminal kinase (JNK)-1, whose activity regulates c-jun transcription and c-Jun phosphorylation (FIG. 3b, right panel and FIG. 3c). These data show that reduction in the inducible expression of these pro-inflammatory genes is mediated through inhibition of c-Jun.

Acute inflammation is a key host response mediated by infiltration of circulating leukocytes, principally neutrophils, from the peripheral blood in order to eliminate pathogens. We assessed the capacity of Dz13 administered by inhalation to modulate acute inflammation in murine lungs challenged with endotoxin. LPS caused a robust increase in neutrophil infiltration in bronchoalveolar lavage fluid 4 h after administration (FIG. 3d). Dz13 administered via the airway only once, localized in cells within the alveolar space and the airways (FIG. 3d) and suppressed this septic response compared to Dz13scr or the vehicle alone in a dose-dependent manner (FIG. 3d).

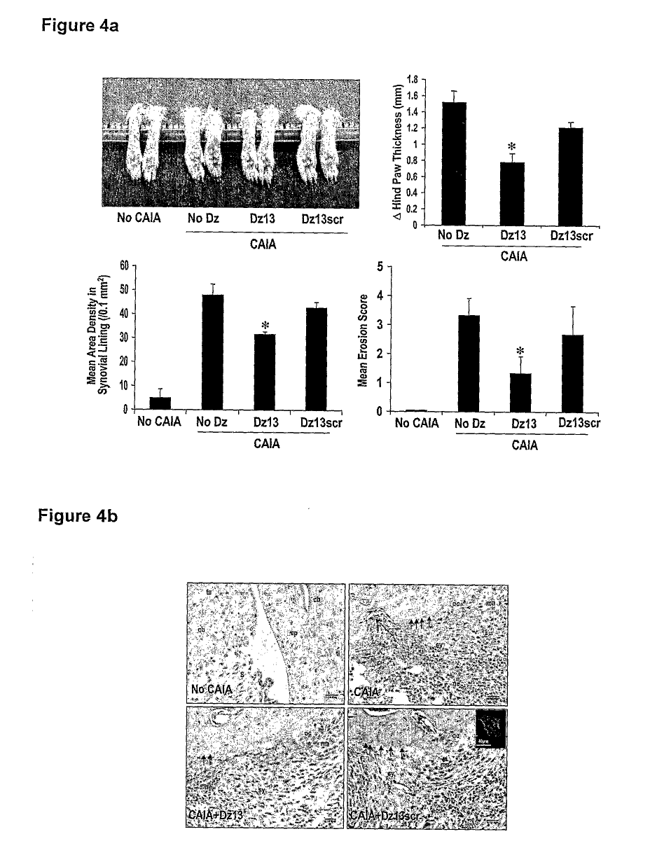

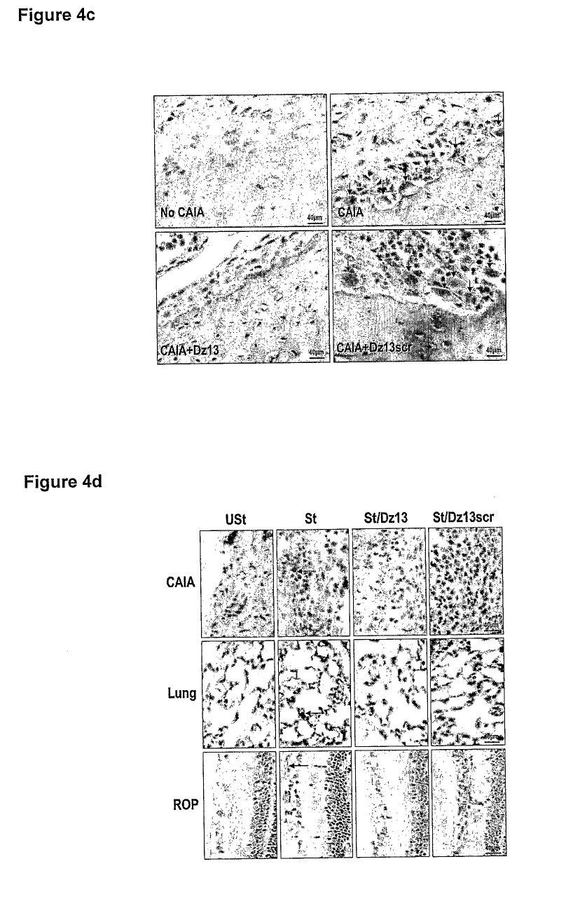

Rheumatoid arthritis is a common and debilitating disease characterized by inflammation of the distal diarthroidial joints. Inflammatory cell infiltration and synovial hyperplasia in these joints contribute to gradual degradation of cartilage and bone, resulting in the loss of normal joint function. The inventors evaluated the anti-inflammatory effects of Dz13 in the murine collagen antibody-induced arthritis model, which has compelling parallels with human inflammatory joint disease (Staines & Wooley (1994) Br J Rheumatol 33, 798-807). Joint inflammation is generated by the systemic administration of a cocktail of four separate collagen monoclonal antibodies together with endotoxin. Dz13 (50 μg) was delivered to the hind paw joint intra-articularly, a clinically-used route of corticosteroid administration 3 days after the induction of arthritis. The DNAzyme inhibited joint thickness (FIG. 4a) and on histologic evaluation, neutrophil accumulation into the synovium and neovascularization (FIG. 4a-c and Table 2). The DNAzyme localized to endothelium and other structures within the joint (FIG. 4b and data not shown). Remarkably, Dz13 also blocked the appearance of multinucleated osteoclast-like cells at the bone surface, and bone erosion (FIG. 4a-c and Table 2). Dz13scr, in contrast, had no effect. These findings indicate the ability of c-Jun DNAzymes to suppress inflammation and bone erosion in this well-established murine model of rheumatoid arthritis. Immunohistochemical analysis revealed that Dz13 inhibited the inducible expression of its target antigen not only in the joint (FIG. 4d), but also in the lung (FIG. 4d) and retina (FIG. 4d), complementing findings in cytokine-treated mesenteric venules (FIG. 3a and Table 1).

TABLE 2

| | | | CAIA + | CAIA + |
|---|---|---|---|---|
| Cell type | No CAIA | CAIA | Dz13 | Dz13scr |
| Neutrophils | − | ++/+++ | + | ++/+++ |
| Multinucleated | +/− | +++ | + | +++ |
| osteoclast-like cells | | | | |
| Macrophages | +/− | ++ | +++ | ++ |
| Fibroblast-like | − | ++ | +++ | +/++ |
| synoviocytes | | | | |
| Neovascularization | − | ++++ | ++/+++ | ++++ |

Numbers of inflammatory cells in the synovial lining if the tibiotarsal joint were evaluated semi-quantitatively by an observer masked to the type of treatments who counted the number of inflammatory cells in 3 randomly selected areas of hematoxylin and eosin-stained sections at 400x. The mean cell count per field and animals in a group was calculated and assigned to a histological grade on a semi-logarithmic scale: − = no cells/field; + = 1-3 cells per field; ++ = 4-10 cells per field; +++ = 11-30 cells per field; ++++ = 31-100 cells per field; +++++ = >101 cells per field.

The inventors have investigated the capacity of catalytic DNA molecules targeting the bZIP transcription factor c-Jun, to perturb vascular permeability and inflammation. Dz13 blocked vascular permeability in the immune complex-triggered passive cutaneous anaphylaxis and the VEGF₁₆₅-induced leakiness models, establishing that c-Jun mediates increased microvascular permeability. Dz13, and an siRNA targeting c-Jun, also inhibited retinal neovascularization. Dz13 completely blocked leukocyte rolling, adhesion and extravasation in the mesenteric venules of rats challenged with IL-1beta. Serial immunohistochemistry and Western blotting revealed the master regulatory role c-Jun plays in the expression of multiple key pro-inflammatory endothelial genes controlling hallmark leukocyte trafficking. Dz13 inhibited E-selectin, VCAM-1, ICAM-1 and VE-cadherin expression, genes regulating the processes of leukocyte rolling, adhesion and extravasation (van Buul Hordijk (2004) Arterioscler Thromb Vasc Biol 24, 824-833). Dz13 suppressed neutrophil infiltration in the airways of mice challenged with LPS in a well-established model of lung sepsis. It also inhibited synovial neutrophil infiltration in the collagen antibody-induced arthritis model. These data indicate, therefore, that vascular permeability (data not shown, refer to Fahmy et al. (2006) Nature Biotechol. 24, 856-863) and inflammation, as well as neovascularization are critically-dependent upon c-Jun. In all systems, Dz13 efficacy was evaluated alongside its scrambled-arm counterpart, Dz13scr (which has identical size, net charge, base composition, and retains the 15-nt catalytic core but is unable to cleave c-Jun mRNA; Khachigian et al. (2002) J. Biol. Chem. 277, 22985-22991) demonstrating c-Jun sequence-specificity. In addition, neither c-Fos, a key partner transcription factor of c-Jun, nor the activated form of its immediate upstream kinase, c-Jun N-terminal kinase-1 (phospho-JNK-1) were affected by Dz13. Dz13 specificity has been demonstrated in previous studies by the inventors, in which Dz13 suppressed levels of c-Jun, but not the zinc finger transcription factor Sp1 in smooth muscle cells of the injured rat carotid artery wall (Khachigian et al. (2002) J. Biol. Chem. 277, 22985-22991). Dz13's site specificity is exclusive for c-Jun mRNA by BLAST analysis. This study demonstrates comparable inhibition by Dz13 and a c-Jun siRNA, each targeting different sites in c-Jun mRNA, and that Dz13 retains its ability to cleave its target sequence after in vivo delivery. The ubiquity of inflammation in a diverse range of human pathologic processes, such as rheumatoid arthritis, asthma, post-infection sepsis, atherosclerotic plaque rupture and erosion, stroke and acute traumatic brain injury, indicates the potential clinical utility of interventional gene-specific strategies targeting c-Jun as primary inhibitors, steroid-spairing agents or as adjuncts.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for treating or inhibiting rheumatoid arthritis in a subject, the method comprising administering to the subject a therapeutically effective amount of a nucleic acid which decreases the level of c-Jun mRNA, c-Jun mRNA translation or nuclear accumulation or activity of c-Jun protein.

2. The method according to claim 1 wherein the nucleic acid is selected from the group consisting of a DNAzyme targeted against c-Jun, a c-Jun antisense oligonucleotide, a ribozyme targeted against c-Jun, and a ssDNA targeted against c-Jun ds DNA such that the ssDNA forms a triplex with the c-Jun dsDNA.

3. The method according to claim 1 wherein the nucleic acid is dsRNA targeted against c-Jun mRNA, a nucleic acid molecule which results in production of dsRNA targeted against c-Jun mRNA or small interfering RNA molecules targeted against c-Jun mRNA.

4. The method according to claim 1 wherein the method is achieved by cleavage of c-Jun mRNA by a sequence-specific DNAzyme.

5. The method according to claim 4 wherein the DNAzyme comprises:
(i) a catalytic domain which cleaves mRNA at a purine:pyrimidine cleavage site;
(ii) a first binding domain contiguous with the 5′ end of the catalytic domain; and
(iii) a second binding domain contiguous with the 3′ end of the catalytic domain;
wherein the binding domains are sufficiently complementary to two regions immediately flanking a purine:pyrimidine cleavage site within the c-Jun mRNA such that the DNAzyme cleaves the c-Jun mRNA.

6. A method according to claim 5 wherein the binding domains have a length of at least 6 nucleotides.

7. A method according to claim 5 wherein both binding domains have a combined total length of at least 14 nucleotides.

8. A method according to claim 5 wherein the binding domain lengths are 9 nucleotides.

9. A method according to claim 5 wherein the catalytic domain has a nucleotide sequence GGCTAGCTACAACGA.

10. A method according to claim 5 wherein the cleavage site is within the region of residues A²⁸⁷ to A¹⁵⁰¹ of the c-Jun mRNA.

11. A method according to claim 5 wherein the cleavage site is within the region of residues U¹²⁹⁶ to G¹⁴⁹⁷ of the c-Jun mRNA.

12. A method according to claim 10 wherein the cleavage site is the GU site corresponding to nucleotides G¹³¹¹U¹³¹².

13. A method according to claim 5 wherein the DNAzyme has the sequence 5′-cgggaggaaGGCTAGCTACAACGAgaggcgttg-3′.

14. A method according to claim 4 wherein the DNAzyme incorporates a 3′-3′ inversion at one or more termini.

15. A method according to claim 2 wherein the c-Jun antisense oligonucleotide comprises a sequence which hybridises to c-Jun within the region of residues U¹²⁹⁶ to G¹⁴⁹⁷.

16. A method according to claim 15 wherein the antisense oligonucleotide has the sequence CGGGAGGAACGAGGCGTTG.

17. A method according to claim 2 wherein the ribozyme cleaves the c-Jun mRNA in the region of residues A²⁸⁷ to A¹⁵⁰¹.

18. A method according to claim 17 wherein the ribozyme cleaves the c-Jun mRNA in the region of residues U¹²⁹⁶ to G¹⁴⁹⁷.

19. A method according to claim 3 wherein the siRNA sense strand is selected from the group consisting of AAGUCAUGAACCACGUUAACA, AAGAACUGCAUGGACCUAACA, CAGCUUCAUGCCUUUGUAA and CAGCUUCCUGCCUUUGUAA.

20. A method according to claim 3 wherein the siRNA is modified to include inverted abasic moieties at the 5′-end and 3′ end of the sense strand and/or a single phosphorothioate linkage between the last two nucleotides at the 3′ end of the antisense strand.

21. A method according to claim 2 wherein the DNAzyme targeted against c-Jun cleaves SEQ ID NO: 9.

22. A method according to claim 2 wherein the c-Jun antisense oligonucleotide, a ribozyme targeted against c-Jun, and a ssDNA targeted against c-Jun dsDNA such that the ssDNA forms a triplex with the c-Jun dsDNA cleave SEQ ID NO: 9.

23. A method according to claim 3 wherein the dsRNA targeted against c-Jun mRNA, a nucleic acid molecule which results in production of dsRNA targeted against c-Jun mRNA or small interfering RNA molecules targeted against c-Jun mRNA cleave SEQ ID NO: 9.

24. A method according to claim 1 wherein administration of the nucleic acid is by intra articular injection.

25. A pharmaceutical composition comprising a nucleic acid which decreases the level of c-Jun mRNA, c-Jun mRNA translation or nuclear accumulation or activity of c-Jun protein, together with a pharmaceutically acceptable carrier, for treating or inhibiting arthritis in a subject.
